(19) United States
(12) Patent Application Publication
(10) Pub. No.: US 2021/0371715 A1
(43) Pub. Date: Dec. 2, 2021

(54) ADHESIVE FOR ENDOSCOPE, CURED PRODUCT THEREOF, ENDOSCOPE, AND METHOD FOR PRODUCING THE SAME

(71) Applicant: FUJIFILM Corporation, Tokyo (JP)

(72) Inventors: Kazushi FURUKAWA, Ashigarakami-gun (JP); Yoshihiro Nakai, Ashigarakami-gun (JP)

(73) Assignee: FUJIFILM Corporation, Tokyo (JP)

(21) Appl. No.: 17/403,981

(22) Filed: Aug. 17, 2021

Related U.S. Application Data
(63) Continuation of application No. PCT/JP2020/006520, filed on Feb. 19, 2020.

(30) Foreign Application Priority Data
Feb. 26, 2019 (JP) 2019-032975

(51) Int. Cl. C09J 163/00; C09J 5/06; C09J 11/06; C08K 5/17; A61B 1/00

(52) U.S. Cl. CPC C09J 163/00; C09J 5/06; A61B 1/00; C08K 5/17; C09J 11/06

(57) ABSTRACT

An adhesive for an endoscope, the adhesive including an epoxy resin including at least one of bisphenol A epoxy resin, bisphenol F epoxy resin, or phenol novolac epoxy resin, a curing component, and an acidic curing acceleration component; a cured product of the adhesive; an endoscope including the cured product fixed; and a method for producing the endoscope.

### CROSS-REFERENCE TO RELATED APPLICATIONS

This application is a Continuation of PCT International Application No. PCT/JP2020/006520 filed on Feb. 19, 2020, which claims priority under 35 U.S.C. § 119 (a) to Japanese Patent Application No. 2019-032975 filed in Japan on Feb. 26, 2019. Each of the above applications is hereby expressly incorporated by reference, in its entirety, into the present application.

### BACKGROUND OF THE INVENTION

### 1. Field of the Invention

The present invention relates to an adhesive for an endoscope, a cured product of the adhesive, an endoscope, and a method for producing the endoscope.

### 2. Description of the Related Art

Endoscopes for observing, for example, the body cavity, the alimentary canal, or the esophagus of the human body are repeatedly used. For this reason, in such an endoscope, the flexible tube forming the insertion section is, after each use, washed or disinfected using a chemical. In particular, in the cases of insertion into highly susceptible regions such as bronchi, cleanliness of the sterilization grade, which is above the disinfection grade, is required. Thus, endoscopes are required to have durability for resisting repeated high-level cleaning treatments; recently, high durability for resisting repeated exposure to sterilization gases such as ethylene oxide gas and hydrogen peroxide gas plasma has come to be required.

The insertion sections of endoscopes are inserted through, for example, the oral cavity or the nasal cavity, into the body. In order to lessen foreign body sensation and pain of the patient during insertion, the insertion sections of endoscopes desirably have reduced diameters. Thus, members constituting the insertion sections are joined together using, instead of bulky members such as screws and bolts, mainly adhesives.

Among adhesives, epoxy adhesives have high usability, and their cured products are excellent in adhesiveness, electrical characteristics, heat resistance, and moisture resistance, for example. For this reason, epoxy adhesives are used in various fields, and use of epoxy adhesives for fixing constituent members of endoscopes has been studied.

For example, JP2008-284191A describes an adhesive for medical devices in which at least one bisphenol epoxy resin of bisphenol A epoxy resin or bisphenol F epoxy resin is used as a base resin and combined with a polyamidoamine curing agent, and the resultant base adhesive is mixed with 1 wt % or more and 30 wt % or less of multi-walled carbon nanotubes having a diameter of 350 nm or less. According to JP2008-284191A, the cured products of this adhesive keep their adhesion strength even after being subjected to various disinfection processes, and also have biocompatibility.

In addition, JP2011-212338A describes a flexible tube for an endoscope, the flexible tube including an adhesiveness-improving layer formed around the outer cover layer and containing a flexible epoxy resin, and an overcoat layer formed around the adhesiveness-improving layer and containing a fluororesin including a vinylidene fluoride unit. According to JP2011-212338A, in the flexible tube for an endoscope, even after autoclave sterilization treatments and hydrogen peroxide plasma sterilization treatments, damage and deterioration of the outer cover are suppressed, and necessary flexibility and protectiveness are maintained.

### SUMMARY OF THE INVENTION

Such an adhesive is used for endoscopes not only in fixing of members, but also as a sealing material for filling the gap between an endoscopic member and a support member with a cured product of the adhesive. For example, glass members disposed at the distal end of the insertion section of an endoscope, such as an illumination window and an observation window, are coated, in the peripheries, with the adhesive and fixed at the distal-end portion, and this adhesive also functions as a sealing material. When the adhesive is used as the sealing material and exposed to sterilization gas, and the sealing material does not have a sufficient gas barrier function, the inside of the tube of the endoscope is exposed to the gas, which results in degradation of the performance of the endoscope. Thus, the adhesive for an endoscope is also required to have a barrier function for sterilization gas.

An object of the present invention is to provide an adhesive for an endoscope, the adhesive being suitable for fixing a constituent member of the endoscope, the adhesive providing, as a result of a curing reaction, a cured product having high performance of gas barrier for sterilization gas, the cured product, even after repeated exposure to sterilization gas, being less likely to deteriorate; and the cured product. Another object of the present invention is to provide an endoscope that is, even after repeated sterilization treatments using sterilization gas, less likely to undergo degradation of the performance, and a method for producing the endoscope.

In order to achieve the above-described objects, the inventors of the present invention performed thorough studies and, as a result, have found that an epoxy adhesive using a combination of an epoxy resin, a curing component for curing the epoxy resin, and further an acidic curing acceleration component achieves the above-described objects. On the basis of such findings, they performed further studies and have accomplished the present invention.

The above-described objects have been achieved by the following means.

[1]

An adhesive for an endoscope, the adhesive including an epoxy resin including at least one of bisphenol A epoxy resin, bisphenol F epoxy resin, or phenol novolac epoxy resin, a curing component, and an acidic curing acceleration component.

[2]

The adhesive for an endoscope according to [1], wherein the acidic curing acceleration component is a compound having a phenolic hydroxy group.

[3]

The adhesive for an endoscope according to [1] or [2], wherein the curing component is a polyamine compound.

[4]

The adhesive for an endoscope according to [3], wherein the curing component is a polyether-polyamine compound.

[5]

The adhesive for an endoscope according to any one of [1] to [4], being used as a sealing material.

[6]

A cured product provided by curing the adhesive for an endoscope according to any one of [1] to [5].

[7]

An endoscope including a constituent member fixed using the cured product according to [6].

[8]

A method for producing an endoscope, the method including fixing a constituent member using the adhesive for an endoscope according to any one of [1] to [5].

In the present invention, when there are a plurality of substituents, linking groups, or the like (hereafter, referred to as substituents etc.) denoted by specific symbols or formulas, or a plurality of substituents etc. are simultaneously defined, the substituents etc. may be the same or different unless otherwise specified. The same applies to the definitions of numbers in substituents etc. When a plurality of substituents etc. are present close to each other (in particular, adjacent to each other), they may be linked together to form a ring unless otherwise specified. In addition, the ring such as an aliphatic ring, an aromatic ring, or a hetero ring may be further fused to form a fused ring unless otherwise specified.

In this Specification, unless otherwise specified, when molecules may have an E or Z configuration for a double bond, molecules employed may have one of these configurations or may be a mixture of these configurations.

It is intended that, unless the present invention is hindered from providing advantages, a partially modified structure is included. Furthermore, compounds that are not clearly described in terms of being substituted or unsubstituted are intended that they may have appropriate substituents unless the present invention is hindered from providing advantages. The same applies to substituents and linking groups. Among such appropriate substituents, preferred substituents in the present invention are substituents selected from Substituent group T described later.

In the descriptions of the present invention, “a value ‘to’ another value” used is intended to include the value and the other value respectively as the lower-limit value and the upper-limit value.

An adhesive for an endoscope according to the present invention provides, as a result of a curing reaction, a cured product having high performance of gas barrier for sterilization gas, and the cured product is less likely to deteriorate even after repeated exposure to sterilization gas. A cured product according to the present invention has high performance of gas barrier for sterilization gas, and is less likely to deteriorate even after repeated exposure to sterilization gas. Thus, an endoscope according to the present invention having this cured product as the fixing material for a constituent member is less likely to undergo degradation of the performance even after repeated sterilization treatments using sterilization gas. Furthermore, a method for producing an endoscope according to the present invention provides an endoscope that is less likely to undergo degradation of the performance even after repeated sterilization treatments using sterilization gas.

### BRIEF DESCRIPTION OF THE DRAWINGS

FIG. 1 is an external view illustrating the configuration of an endoscope according to an embodiment of the present invention;

FIG. 2 is a partial sectional view illustrating the configuration of the insertion section of the endoscope in FIG. 1;

FIG. 3 is an external perspective view illustrating the distal-end portion of the insertion section; and

FIG. 4 is a cutaway partial sectional view illustrating the distal-end portion in which the sections of the lenses and the prism are not hatched.

### DESCRIPTION OF THE PREFERRED EMBODIMENTS

### Adhesive for Endoscope

Preferred embodiments of an adhesive for an endoscope according to the present invention will be described.

An adhesive for an endoscope according to the present invention (hereafter, also referred to as “adhesive according to the present invention”) includes (A) an epoxy resin, (B) a curing component, and (C) an acidic curing acceleration component, wherein the (A) epoxy resin includes at least one of bisphenol A epoxy resin, bisphenol F epoxy resin, or phenol novolac epoxy resin.

The (A) epoxy resin (hereafter, also simply referred to as “Component (A)”) is the base resin of the adhesive. The (B) curing component (hereafter, also simply referred to as “Component (B)”) is a component that reacts with the epoxy resin to cure the adhesive. The (C) acidic curing acceleration component (hereafter, also simply referred to as “Component (C)”) is a component that activates epoxy groups in the epoxy resin, to accelerate the curing reaction of the epoxy resin caused by Component (B) and simultaneously to cause formation of a self-polymerization product of the epoxy resin itself. In an adhesive according to the present invention, the formulation mass of the (C) curing acceleration component is ordinarily less than that of the (B) curing component.

An adhesive according to the present invention, as long as it includes the above-described components, is not limited in terms of form. For example, an adhesive for an endoscope according to the present invention may have a form containing a mixture of Components (A) to (C) above (one-component type), or may include Components (A) to (C) above such that one or more components among Components (A) to (C) are separated from the other component (two-component type). Alternatively, an adhesive for an endoscope according to the present invention may include Components (A) to (C) such that Components (A) to (C) are separated from each other (three-component type). All these forms fall within the scope of an adhesive according to the present invention.

In this Specification, descriptions of the contents of components in adhesives or definitions of, in the present invention, the contents of components in adhesives are intended that, in the cases of the form of the two-component type or the three-component type, Components (A) to (C) are mixed at the time of use such that the components satisfy the desired contents in the mixtures. In other words, in the cases of the form of the two-component type or the three-component type, in a state where the components are separated, the contents of Components (A) to (C) do not necessarily satisfy the contents described in this Specification or the contents defined in the present invention. Stated another way, the form of the two-component type or the three-component type satisfies, at the time when Components (A) to (C) are mixed together for use, the contents described in this Specification or the contents defined in the present invention.

When an adhesive for an endoscope according to the present invention is of the one-component type or even the two-component type or the like and includes a mixture of components that react with each other (for example, a mixture of an epoxy resin and a curing component), in order to prevent or sufficiently suppress the reaction between the components to keep a state in which the components are maintained with stability, the adhesive is preferably stored at such a low temperature that the reaction substantially does not occur. For example, the storage can be performed at −20° C. or less, preferably −30° C. or less, more preferably −40° C. or less, still more preferably −50° C. or less. The storage can be performed under light-tight conditions as needed.

An adhesive according to the present invention may include, unless the present invention is hindered from providing advantages, for example, a solvent, a plasticizer, an adhesiveness improver (such as a silane coupling agent), a surfactant, a coloring agent (such as a pigment or a dye), an anti-weathering agent, an antioxidant, a heat stabilizer, a lubricant, an antistatic agent, a brightening agent, a release agent, a conductive agent, a viscosity modifier, a filler (such as silica or calcium carbonate), a thixotropy imparting agent, a diluent, and a flame retardant.

An adhesive according to the present invention provides, as a result of a curing reaction, a cured product that has high gas barrier performance. In addition, it is less likely to deteriorate even after being subjected to repeated sterilization treatments using sterilization gas, and sufficiently maintains high gas barrier performance and adhesiveness. The reason for this is not clarified, but is inferred as follows.

An adhesive according to the present invention includes, in addition to an epoxy resin and a curing component, an acidic curing acceleration component, to thereby activate the epoxy groups, to accelerate the curing reaction. At this time, the self-polymerization reaction of the epoxy resin itself also occurs, so that the self-polymerization product of the epoxy resin is introduced into the cured product. The self-polymerization product of the epoxy resin has a molecular structure having a high degree of crosslinking, so that the cured product has high gas barrier performance and is less likely to deteriorate even after exposure to sterilization gas. In other words, even after repeated exposure to sterilization gas, the gas barrier performance or adhesiveness is less likely to degrade. In addition, the epoxy resin in an adhesive according to the present invention has a high proportion of aromatic rings, which inferentially contributes to improvement in the gas barrier performance.

On the other hand, as known curing acceleration components other than acidic curing acceleration components, there are tertiary amine compounds and phosphine compounds, for example. In the case of using such a curing acceleration component other than acidic curing acceleration components, epoxy groups are not activated, so that the curing reaction of the epoxy resin caused by the curing component proceeds slowly, and the resultant cured product has constituent components most of which are self-polymerization reaction products of epoxy groups. When this cured product having constituent components most of which are self-polymerization reaction products of epoxy groups is sterilized, the gas barrier performance of the cured product is degraded. This is inferentially caused because the sterilization embrittles the cured product to cause slight cracking therein and the cracking allows the gas to pass therethrough.

An adhesive according to the present invention is used to fix various constituent members of an endoscope (endoscope constituent members). Specifically, an adhesive according to the present invention is used to bond an endoscope constituent member to another endoscope constituent member, to thereby fix the endoscope constituent member to the other endoscope constituent member. The adhesive having been used for fixing the endoscope constituent member turns into a cured product to form a bonding portion of the endoscope.

The member fixed using an adhesive according to the present invention is not particularly limited, and preferred examples include metal members, glass members, and resin members. Such an endoscope constituent member is “fixed” by bonding the endoscope constituent member to another constituent member (support member) of the endoscope. Incidentally, the support member may be, for example, the tube wall of the endoscope or an immovable member fixed on the tube wall or the like, or may be a member that is movable to another relative position within the endoscope, such as the tube. In the present invention, the term “fix” is used with meanings including filling, with a cured product of an adhesive, the gap between an endoscope constituent member and a support member into which the member is incorporated, namely, sealing.

Hereinafter, constituent components of an adhesive according to the present invention will be described.

### (A) Epoxy Resin

An adhesive according to the present invention includes, as Component (A), an epoxy resin; this epoxy resin includes at least one of bisphenol A epoxy resin, bisphenol F epoxy resin, or phenol novolac epoxy resin. An adhesive according to the present invention may include one or two or more epoxy resins selected from the group consisting of bisphenol A epoxy resin, bisphenol F epoxy resin, and phenol novolac epoxy resin.

Relative to the total amount of epoxy resin included in an adhesive according to the present invention, the percentage of the total amount of the bisphenol A epoxy resin, the bisphenol F epoxy resin, and the phenol novolac epoxy resin is preferably 70 mass % or more, preferably 80 mass % or more, more preferably 90 mass % or more. More preferably, the epoxy resin included in an adhesive according to the present invention is at least one of bisphenol A epoxy resin, bisphenol F epoxy resin, or phenol novolac epoxy resin.

The epoxy resin included in an adhesive according to the present invention preferably has an epoxy equivalent of 10 to 1000, more preferably 50 to 500, still more preferably 80 to 400, particularly preferably 100 to 300. The epoxy resin included in an adhesive according to the present invention ordinarily has two or more epoxy groups in a single molecule.

The epoxy equivalent is a value obtained by dividing the molecular weight of an epoxy compound by the number of moles of the epoxy groups of the epoxy compound.

The bisphenol A epoxy resin usable for an adhesive according to the present invention is not particularly limited, and such resins commonly used as the base resins of epoxy adhesives can be widely employed. Preferred specific examples include bisphenol A diglycidyl ethers (jER 825, jER 828, and jER 834 (all of which are trade names), manufactured by Mitsubishi Chemical Corporation) and a bisphenol A propoxylate diglycidyl ether (manufactured by Sigma-Aldrich Corporation).

The bisphenol F epoxy resin usable for an adhesive according to the present invention is not particularly limited, and such resins commonly used as the base resins of epoxy adhesives can be widely employed. Preferred specific examples include a bisphenol F diglycidyl ether (trade name: EPICLON 830, manufactured by DIC Corporation) and 4,4′-methylenebis(N,N-diglycidylaniline).

The phenol novolac epoxy resin usable for an adhesive according to the present invention is not particularly limited, and such resins commonly used as the base resins of epoxy adhesives can be widely employed. Such a phenol novolac epoxy resin is, for example, commercially available as product number: 406775 from Sigma-Aldrich Corporation.

The content of the epoxy resin included in an adhesive according to the present invention can be set at 5 to 90 mass %, more preferably 10 to 75 mass %.

### (B) Curing Component

An adhesive according to the present invention contains, as Component (B), one or two or more curing components. The curing component included in an adhesive according to the present invention is not particularly limited, and various curing agents known as curing components for epoxy adhesives can be employed. Examples include acid anhydride compounds, imidazole compounds, phosphorus-containing compounds, polythiol compounds, dicyandiamide compounds, phenolic compounds, and polyamine compounds.

An adhesive according to the present invention, from the viewpoint of sufficiently maintaining the gas barrier performance even after sterilization treatments, preferably includes, as Component (B), at least one of a polyamine compound, a polythiol compound, or an acid anhydride compound. From the viewpoint of formation of a crosslinking structure more stable against disinfectants used for endoscopes such as peracetic acid, the adhesive more preferably includes, as Component (B), a polyamine compound. Hereinafter, the curing components usable as Component (B) will be described in detail.

### (1) Polyamine Compound

An adhesive according to the present invention preferably contains, as Component (B), one or two or more polyamine compounds.

Such a polyamine compound is a compound that has, in a single molecule, two or more amino groups having an active hydrogen. This polyamine compound preferably has an unsubstituted amino group (—NH₂), more preferably has two or more unsubstituted amino groups. This polyamine compound is still more preferably a primary polyamine compound (a polyamine compound in which all the amino groups are unsubstituted amino groups). For an adhesive according to the present invention, polyamine compounds that exert a curing action in epoxy adhesives can be widely employed.

In a single molecule of the polyamine compound, the number of amino groups having an active hydrogen is preferably 2 to 10, more preferably 2 to 8, still more preferably 2 to 6, still more preferably 2 to 4, particularly preferably 2 or 3. In particular, at least one selected from the group consisting of diamine compounds and triamine compounds can be suitably used.

The polyamine compound preferably has an active-hydrogen equivalent (the equivalent of the active hydrogens of amino groups) of 10 to 2000, more preferably 20 to 1000, still more preferably 30 to 900, still more preferably 40 to 800, still more preferably 60 to 700, particularly preferably 65 to 600.

The active-hydrogen equivalent is a value obtained by dividing the molecular weight of the polyamine compound by the number of moles of active hydrogens of amino groups in the polyamine compound (it means the molecular weight per active hydrogen of amino groups in the polyamine compound).

The polyamine compound preferably has a molecular weight of 100 to 6000, more preferably 100 to 3000. When the polyamine compound is a polymer (for example, in the case of having a polyoxyalkylene group described later), such a molecular weight is a number-average molecular weight.

The polyamine compound preferably has a form in which two or more amino groups are bonded to each other via a group selected from the group consisting of aliphatic hydrocarbon groups, cyclic hydrocarbon groups, aromatic hydrocarbon groups, and heterocyclic groups, or a group of a combination of the foregoing. Such a group may have, in a carbon-carbon bond, a hetero atom such as an oxygen atom, a nitrogen atom, or a sulfur atom (preferably, an oxygen atom).

The polyamine compound, from the viewpoint of being less likely to react with radicals generated by hydrogen peroxide plasma treatments, also preferably does not include, in a carbon-carbon bond, a hetero atom such as an oxygen atom, a nitrogen atom, or a sulfur atom (preferably an oxygen atom). When the polyamine compound does not include a hetero atom in a carbon-carbon bond, the group that bonds together two or more amino groups is more preferably a chain aliphatic hydrocarbon group, and the chain aliphatic hydrocarbon group may have a branch. In such a chain aliphatic hydrocarbon group that may have a branch, the number of carbon atoms is preferably 4 to 50, more preferably 4 to 12, still more preferably 6 to 12.

The polyamine compound, from the viewpoint of imparting further flexibility to the cured product to achieve sturdier properties, also preferably has, in the molecule, a chain alkylene group or an oxyalkylene structure, more preferably has a polyoxyalkylene structure.

The polyamine compound having a chain alkylene group is preferably an alkylenediamine compound. The polyamine compound having a polyoxyalkylene structure (hereafter, also referred to as “polyether-polyamine compound”) is more preferably a polyoxyalkylenediamine compound or a polyoxyalkylenetriamine compound.

The chain alkylene group may be linear or branched, and the number of carbon atoms is preferably 1 to 20, more preferably 5 to 12. Specific examples of the alkylene group include methylene, ethylene, hexamethylene, 2,4,4-trimethylhexamethylene, 2-methylpentamethylene, and dodecamethylene.

The alkylene group of the oxyalkylene structure may be a linear alkylene group or an alkylene group having a branch. In the alkylene group of the oxyalkylene structure, the number of carbon atoms is preferably 1 to 10, more preferably 2 to 6, still more preferably 2 to 4.

The oxyalkylene structure is more preferably an oxyethylene group or an oxypropylene group.

When the polyamine compound of Component (B) has a polyoxyalkylene structure, the plurality of oxyalkylene groups constituting the polyoxyalkylene structure may be the same or different. The average repeating number of the oxyalkylene groups in the polyoxyalkylene structure is preferably 2 to 1000, more preferably 3 to 500. The average repeating number is also preferably 2 to 100, also preferably 2 to 50, also preferably 2 to 35, also preferably 2 to 25. The polyamine compound of Component (B) may have a plurality of polyoxyalkylene structures.

The polyamine compound, from the viewpoint of forming an intramolecular hydrogen bond to provide a material (cured product) having sturdier properties, also preferably has, in the molecule, an amide bond (—NH—CO—), more preferably has a polyamide bond.

The polyamine compound having a polyamide bond (hereafter, also referred to as “polyamide polyamine compound”) is preferably a polyamide diamine compound.

The average number of amide bonds per molecule of the polyamide polyamine compound is preferably 2 to 50, more preferably 5 to 30, still more preferably 5 to 20.

The linking group that links together the plurality of amide bonds is not particularly limited, and examples include saturated or unsaturated aliphatic hydrocarbon groups and aromatic hydrocarbon groups. When the polyamide polyamine compound has a plurality of linking groups that link together amide bonds, the plurality of linking groups may be the same or different.

In an adhesive according to the present invention, the polyamine compound preferably has a polyoxyalkylene structure. When the polyamine compound is a compound having a polyoxyalkylene structure, the cured product has high durability for sterilization treatments, and generation of internal cracking of the cured product due to deterioration is suppressed, which inferentially results in maintaining of the gas barrier performance even after sterilization treatments.

Specific preferred examples of the polyamine compound usable for the present invention are as follows. The numbers at the parentheses are the average repeating numbers of the repeating units within the parentheses.

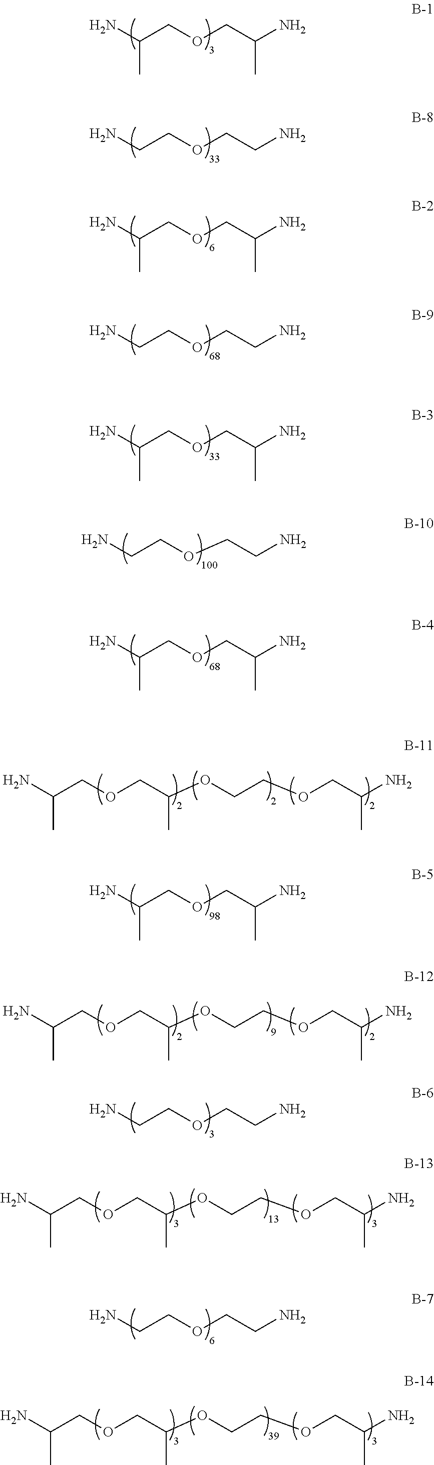

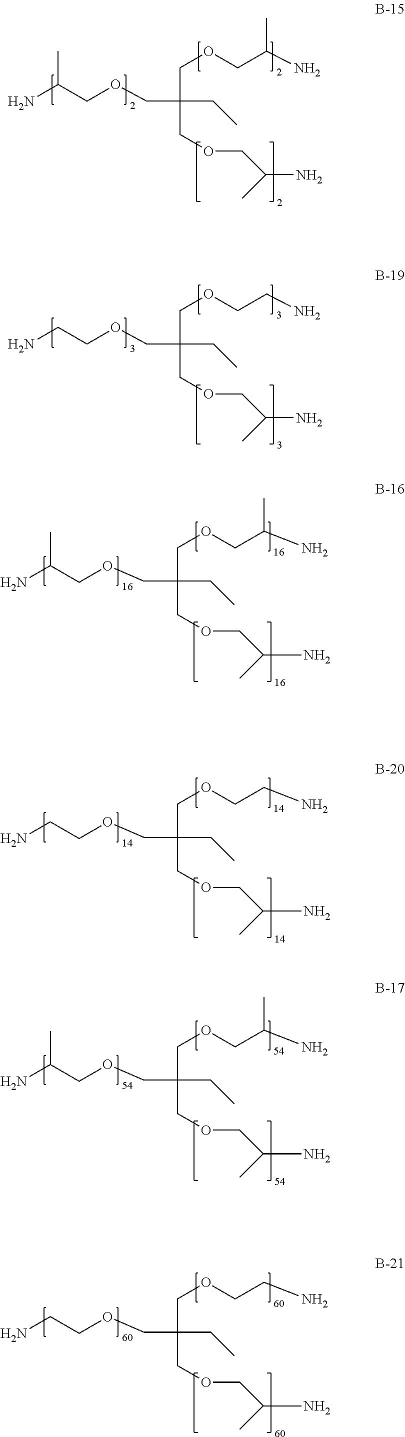

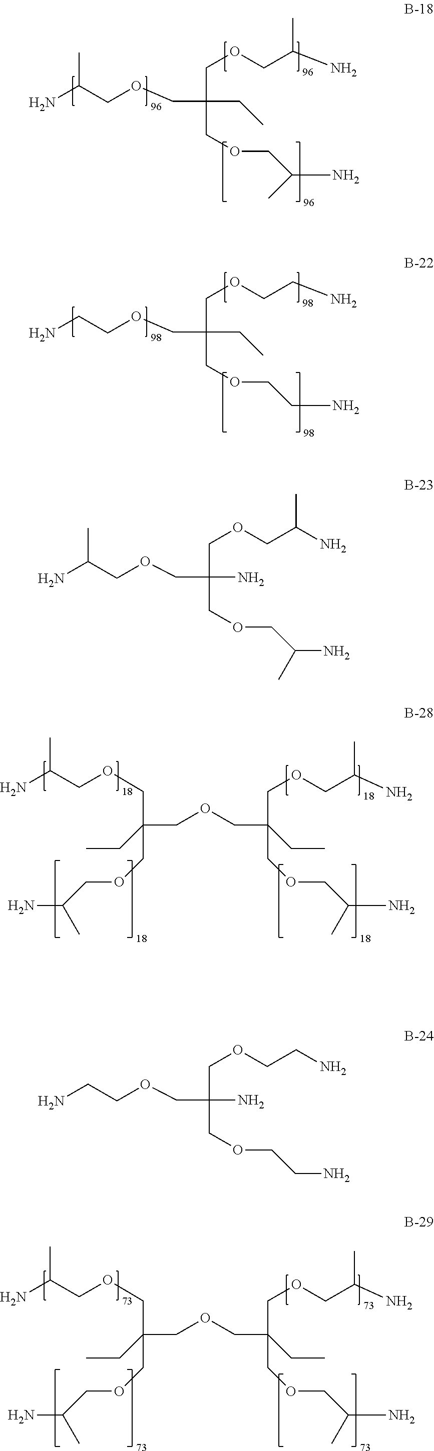

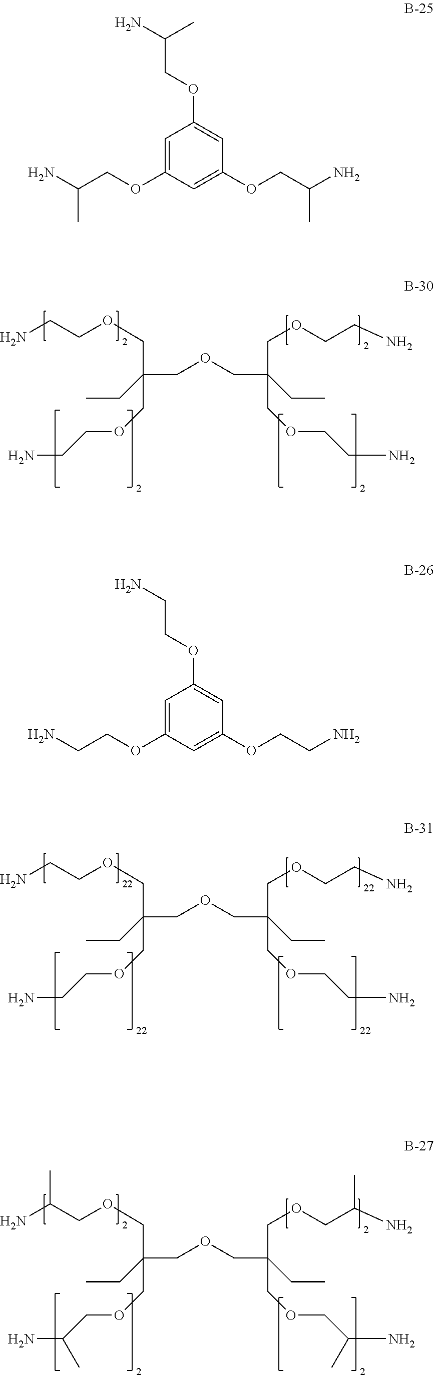

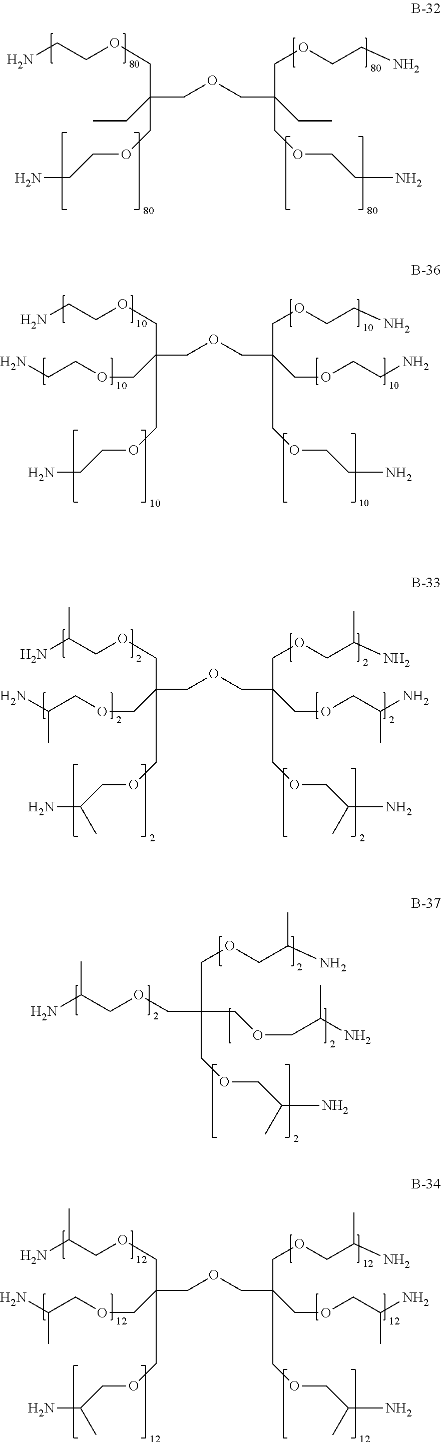

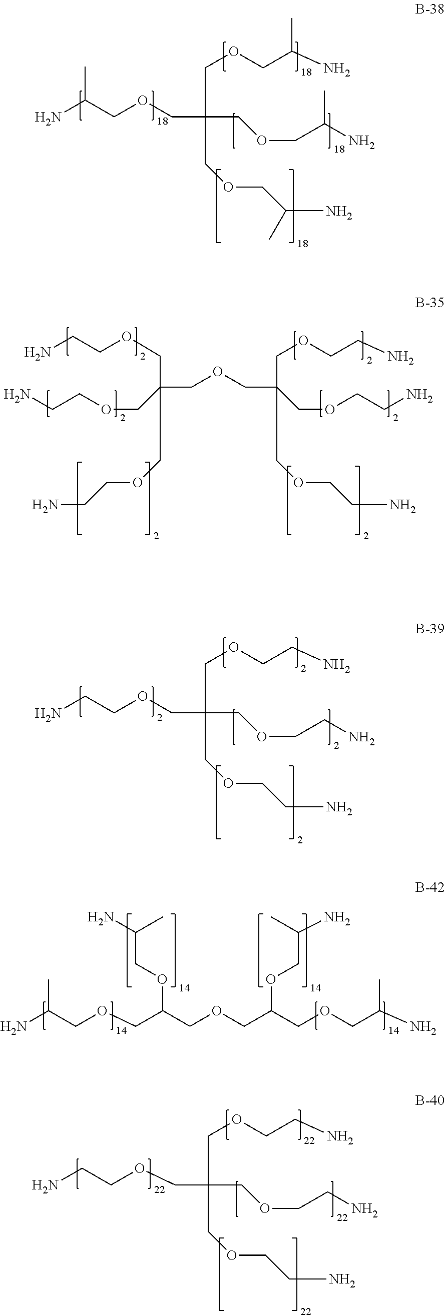

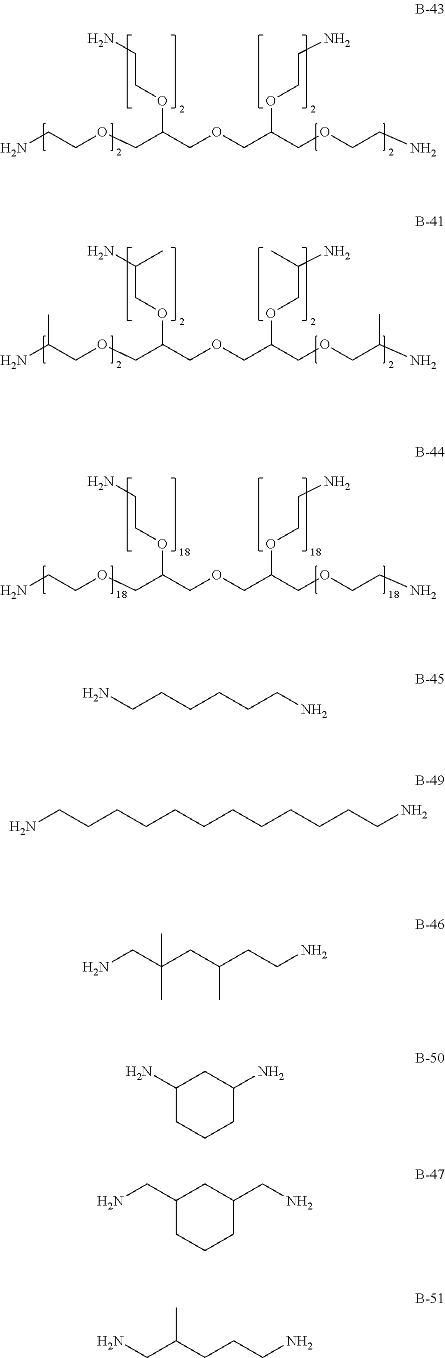

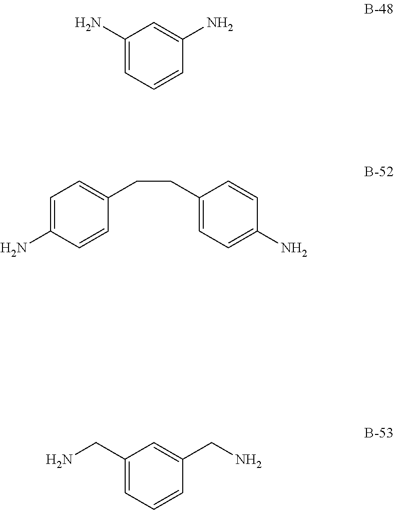

The above-described polyamine compounds can be synthesized in the standard manner. Alternatively, commercially available products may be used.

### (2) Acid Anhydride Compound

An adhesive according to the present invention preferably contains, as Component (B), one or two or more acid anhydride compounds.

Such an acid anhydride compound inferentially causes a copolycondensation reaction with epoxy groups of the epoxy resin serving as Component (A), to cure the epoxy resin. In the present invention, “acid anhydride” means carboxylic anhydride.

Examples of the acid anhydride compound include alicyclic dicarboxylic anhydrides and aromatic cyclic dicarboxylic anhydrides.

Such an alicyclic dicarboxylic anhydride is a compound having a structure in which the carboxy groups at two adjacent carbon atoms constituting the aliphatic ring have undergone dehydration-condensation. Such an aromatic cyclic dicarboxylic anhydride is a compound having a structure in which the carboxy groups at two adjacent carbon atoms constituting the aromatic ring have undergone dehydration-condensation.

The aliphatic ring may or may not partially have, between atoms constituting the ring, a carbon-carbon unsaturated bond, but preferably does not have the carbon-carbon unsaturated bond. This aliphatic ring may be a bridged ring such as a bicyclo ring.

These aliphatic ring and aromatic ring are preferably five-membered rings or six-membered rings.

In particular, these aliphatic ring. and aromatic ring are preferably monocyclic, more preferably six-membered rings.

These aliphatic ring and aromatic ring may have one or two or more substituents.

Preferred examples of the substituents that may be present at the aliphatic ring and the aromatic ring include alkyl groups, alkoxy groups, acyl groups, alkoxycarbonyl groups, aryl groups, and carboxy groups. The number of carbon atoms of such an alkyl group is preferably 1 to 10, more preferably 1 to 6, still more preferably 1 to 3, particularly preferably 1 or 2. The number of carbon atoms of such an alkoxy group is preferably 1 to 10, more preferably 1 to 6, still more preferably 1 to 3, particularly preferably 1 or 2. The number of carbon atoms of such an acyl group (including an alkylcarbonyl group and an arylcarbonyl group) is preferably 1 to 30, more preferably 1 to 20, still more preferably 1 to 10. The number of carbon atoms of such an alkoxycarbonyl group is preferably 2 to 30, more preferably 2 to 20, still more preferably 2 to 15, particularly preferably 2 to 4. The number of carbon atoms of such an aryl group is preferably 6 to 20, more preferably 6 to 15, still more preferably 6 to 12, particularly preferably 6.

Among such substituents, two substituents close to each other may be linked to form a ring. Such a ring formed by two substituents linked together is preferably a monocyclic five-membered ring or six-membered ring; two carboxy groups bonded to adjacent atoms constituting the ring are also preferably linked together to form an acid anhydride structure.

A plurality of monovalent or di- or higher valent groups obtained by removing one or two or more hydrogen atoms from such a substituent are also preferably bonded together to form a compound having two or three or more acid anhydride structures in the compound.

The number of the substituents of such an aliphatic ring or aromatic ring is preferably 0 or 1.

The acid anhydride compound is preferably an alicyclic dicarboxylic anhydride.

The number of acid anhydride structures in a molecule of such an acid anhydride compound is preferably 1 to 3, more preferably 1 or 2, still more preferably 1.

Such an acid anhydride compound preferably has a molecular weight of 90 to 800, more preferably 100 to 300.

The acid anhydride compound preferably includes at least one selected from the group consisting of phthalic anhydride, trimellitic anhydride, pyromellitic anhydride, benzophenonetetracarboxylic anhydride, ethylene glycol bisanhydrotrimellitate, glycerol trisanhydrotrimellitate, maleic anhydride, tetrahydrophthalic anhydride, methyltetrahydrophthalic anhydride, methylbutenyltetrahydrophthalic anhydride, hexahydrophthalic anhydride, methylhexahydrophthalic anhydride, succinic anhydride, octenylsuccinic anhydride, dodecenylsuccinic anhydride, methylcyclohexenedicarboxylic anhydride, methylbicyclo[2.2.1]heptane-2,3-dicarboxylic anhydride, and bicyclo[2.2.1]heptane-2,3-dicarboxylic anhydride (hereafter, these acid anhydride compounds will be collectively referred to as Acid anhydride Z). Thus, the acid anhydride compound serving as Component (B) above preferably includes at least one acid anhydride compound selected from Acid anhydride Z.

Acid anhydride Z above is more preferably an acid anhydride compound selected from the group consisting of trimellitic anhydride, hexahydrophthalic anhydride, methylhexahydrophthalic anhydride, succinic anhydride, methylbicyclo[2.2.1]heptane-2,3-dicarboxylic anhydride, bicyclo[2.2.1]heptane-2,3-dicarboxylic anhydride, benzophenonetetracarboxylic anhydride, glycerol trisanhydrotrimellitate, and octenylsuccinic anhydride.

When the acid anhydride compound serving as Component (B) above includes an acid anhydride compound selected from Acid anhydride Z above, this Component (B), unless the present invention is hindered from providing advantages, may include an acid anhydride compound other than Acid anhydride Z. In this case, relative to all the acid anhydride compounds included as Component (B), the percentage of the total amount of the acid anhydride compound selected from Acid anhydride Z is preferably 50 mass % or more, more preferably 70 mass % or more, still more preferably 80 mass % or more, particularly preferably 90 mass % or more. Alternatively, all the acid anhydride compounds included as Component (B) above are also preferably acid anhydride compounds selected from Acid anhydride Z above.

The acid anhydride compound that can be included as Component (B) above, from the viewpoint of transparency of the cured product, preferably does not include nitrogen atoms.

The following are specific examples of acid anhydride compounds usable for the present invention; however, the present invention is not limited to these.

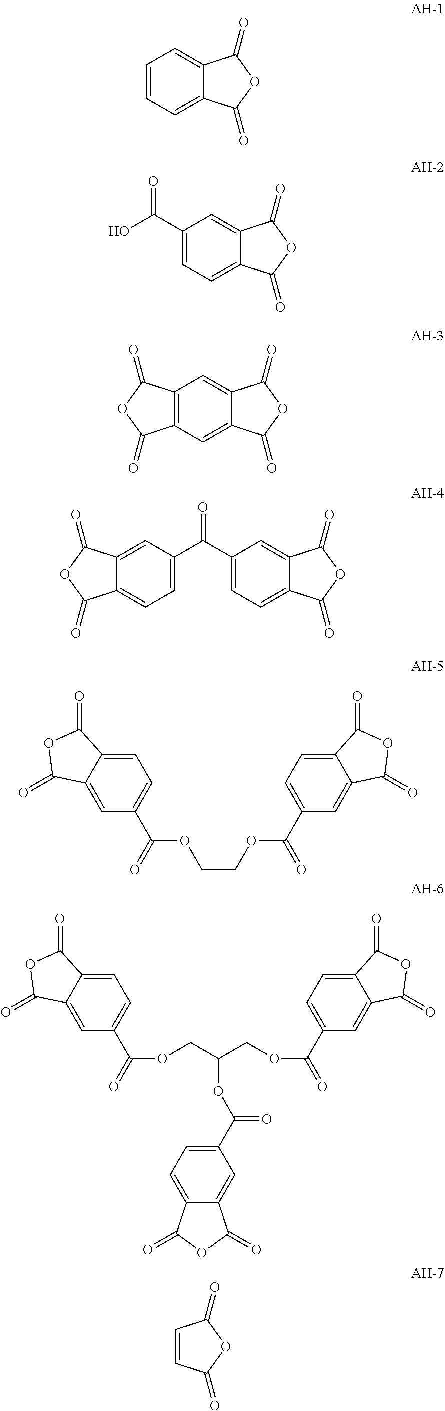

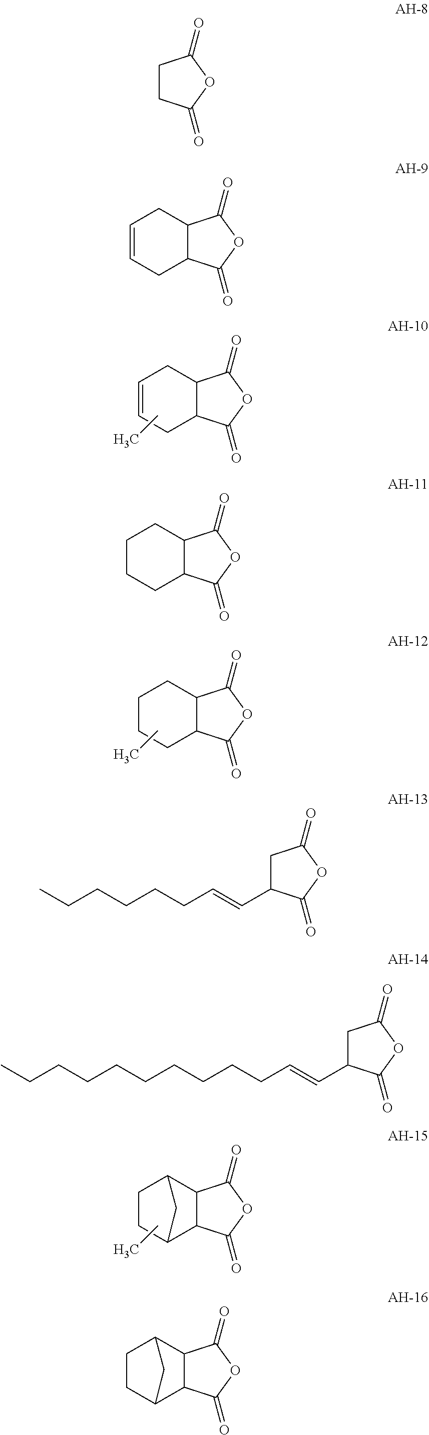

The above-described acid anhydride compounds can be synthesized in the standard manner. Alternatively, commercially available products may be used.

### (3) Thiol Compound

An adhesive according to the present invention preferably contains, as Component (B), one or two or more thiol compounds.

Such a thiol compound is a compound that has at least two moieties represented by General formula (1) below, or at least two moieties represented by General formula (2) below. Such a thiol compound that has a structure having 3 to 10 (preferably 3 to 6) moieties represented by General formula (1) below, or 3 to 10 (preferably 3 to 6) moieties represented by General formula (2) below is preferred from the viewpoint that the cured product has increased crosslinking density to have further improved chemical resistance. On the other hand, such a thiol compound that has a structure having two moieties represented by General formula (1) below, or two moieties represented by General formula (2) below is preferred from the viewpoint that the resultant cured product is relatively flexible to exhibit impact resistance.

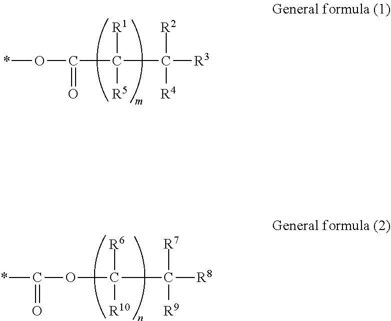

In General formula (1), one of R¹ to R⁵ represents a sulfanyl group (thiol group), and the others each independently represent a hydrogen atom, an alkyl group having 1 to 10 carbon atoms, or an aryl group having 6 to 14 carbon atoms; m represents an integer of 0 to 2. When m is 2, two R¹'s may be the same or different, and two R⁵'s may be the same or different. * denotes the point of attachment in the thiol compound.

In General formula (2), one of R⁶ to R¹⁰ represents a sulfanyl group, and the others each independently represent a hydrogen atom, an alkyl group having 1 to 10 carbon atoms, or an aryl group having 6 to 14 carbon atoms; n represents an integer of 0 to 2. When n is 2, two R⁶'s may be the same or different, and two R¹⁰ 's may be the same or different. * denotes the point of attachment in the thiol compound.

The alkyl group having 1 to 10 carbon atoms may be linear or branched, and examples include methyl, ethyl, propyl, i-propyl, butyl, i-butyl, t-butyl, hexyl, and octyl. Of these, methyl or ethyl is preferred.

Specific examples of the aryl group having 6 to 14 carbon atoms include phenyl and naphthyl.

m is preferably 0 or 1.

n is preferably 0 or 1.

The moiety represented by General formula (1) above is preferably a moiety represented by General formula (3) below.

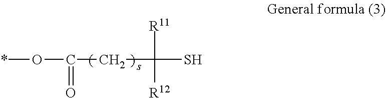

In General formula (3), R¹¹ and R¹² each independently represent a hydrogen atom or an alkyl group having 1 to 10 carbon atoms, and s represents an integer of 0 to 2. * denotes the point of attachment in the thiol compound.

At least one of R¹¹ or R¹² preferably represents an alkyl group having 1 to 10 carbon atoms.

The alkyl groups having 1 to 10 carbon atoms represented by R¹¹ and R¹² have the same definitions and preferred examples as in the above-described alkyl group that can be employed as R¹ in General formula (1).

s is preferably 0 or 1, more preferably 1.

The thiol compound is preferably an ester of a compound represented by General formula (4) below and a polyfunctional alcohol.

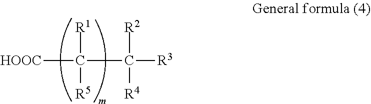

In General formula (4), R¹ to R⁵ and m respectively have the same definitions and preferred examples as in R¹ to R⁵ and m in General formula (1) above.

The compound represented by General formula (4) is preferably a compound represented by General formula (5) below.

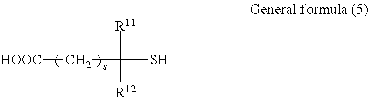

In General formula (5), R¹¹, R¹², and s respectively have the same definitions and preferred examples as in R¹¹, R¹², and s in General formula (3) above.

Specific examples of the compound represented by General formula (4) above include 2-mercaptopropionic acid, 3-mercaptopropionic acid, 3-mercaptobutanoic acid, 2-mercaptoisobutanoic acid, 3-mercapto-3-phenylpropionic acid, 3-mercaptoisobutyric acid, 2-mercapto-3-methylbutyric acid, 3-mercapto-3-methylbutyric acid, 3-mercaptovaleric acid, and 3-mercapto-4-methylvaleric acid.

The polyfunctional alcohol is preferably an alcohol having 2 to 10 functionality (polyol having 2 to 10 hydroxy groups), more preferably 2 to 8 functionality, particularly preferably 2 to 6 functionality.

Specific examples of the polyfunctional alcohol include alkylene glycols (the alkylene groups preferably have 2 to 10 carbon atoms, and the alkylene groups may be linear or branched), diethylene glycol, glycerol, dipropylene glycol, trimethylolpropane, pentaerythritol, and dipentaerythritol.

Examples of the alkylene glycols include ethylene glycol, trimethylene glycol, 1,2-propane glycol, 1,2-butanediol, 1,3-butanediol, 2,3-butanediol, and tetramethylene glycol.

Preferred examples of the polyfunctional alcohol include alkylene glycols having an alkylene main chain having 2 carbon atoms, such as ethylene glycol, 1,2-propane glycol, and 1,2-butanediol, trimethylolpropane, and pentaerythritol.

The following are specific examples of thiol compound usable in the present invention; however, the present invention is not limited to these.

Specific examples include bis(1-mercaptoethyl) phthalate, bis(2-mercaptopropyl) phthalate, bis(3-mercaptobutyl) phthalate, bis(3-mercaptoisobutyl) phthalate, ethylene glycol bis(3-mercaptopropionate), ethylene glycol bis(3-mercaptobutyrate), propylene glycol bis(3-mercaptobutyrate), diethylene glycol bis(3-mercaptobutyrate), tetraethylene glycol bis(3-mercaptopropionate), butanediol bis(3-mercaptobutyrate), octanediol bis(3-mercaptobutyrate), trimethylolpropane tris(3-mercaptobutyrate), trimethylolpropane tris(3-mercaptopropionate), dipentaerythritol hexakis(3-mercaptopropionate), pentaerythritol tetrakis(3-mercaptopropionate), pentaerythritol tetrakis(3-mercaptobutyrate), dipentaerythritol hexakis(3-mercaptobutyrate), ethylene glycol bis(2-mercaptopropionate), propylene glycol bis(2-mercaptopropionate), diethylene glycol bis(2-mercaptopropionate), butanediol bis(2-mercaptopropionate), octanediol bis(2-mercaptopropionate), trimethylolpropane tris(2-mercaptopropionate), pentaerythritol tetrakis(2-mercaptopropionate), dipentaerythritol hexakis(2-mercaptopropionate), ethylene glycol bis(3-mercaptoisobutyrate), propylene glycol bis(3-mercaptoisobutyrate), diethylene glycol bis(3-mercaptoisobutyrate), butanediol bis(3-mercaptoisobutyrate), octanediol bis(3-mercaptoisobutyrate), trimethylolpropane tris(3-mercaptoisobutyrate), pentaerythritol tetrakis(3-mercaptoisobutyrate), dipentaerythritol hexakis(3-mercaptoisobutyrate), ethylene glycol bis(2-mercaptoisobutyrate), propylene glycol bis(2-mercaptoisobutyrate), diethylene glycol bis(2-mercaptoisobutyrate), butanediol bis(2-mercaptoisobutyrate), octanediol bis(2-mercaptoisobutyrate), trimethylolpropane tris(2-mercaptoisobutyrate), pentaerythritol tetrakis(2-mercaptoisobutyrate), dipentaerythritol hexakis(2-mercaptoisobutyrate), ethylene glycol bis(4-mercaptovalerate), propylene glycol bis(4-mercaptoisovalerate), diethylene glycol bis(4-mercaptovalerate), butanediol bis(4-mercaptovalerate), octanediol bis(4-mercaptovalerate), trimethylolpropane tris(4-mercaptovalerate), pentaerythritol tetrakis(4-mercaptovalerate), dipentaerythritol hexakis(4-mercaptovalerate), ethylene glycol bis(3-mercaptovalerate), propylene glycol bis(3-mercaptovalerate), diethylene glycol bis(3-mercaptovalerate), butanediol bis(3-mercaptovalerate), octanediol bis(3-mercaptovalerate), trimethylolpropane tris(3-mercaptovalerate), pentaerythritol tetrakis(3-mercaptovalerate), dipentaerythritol hexakis(3-mercaptovalerate), 1,4-bis(3-mercaptobutyryloxy)butane, 1,3,5-tris(3-mercaptobutyloxyethyl)-1,3,5-triazine-2,4,6(1H,3H,5H)-trione, and tris[(3-mercaptopropionyloxy)ethyl] isocyanurate.

From the viewpoint of having less odor, desired viscosities, and high compatibility with the epoxy resin serving as Component (A), and providing the handleability of a mixture obtained by mixing Component (A) and Component (B), the thiol compound is preferably at least one of 1,4-bis(3-mercaptobutyryloxy)butane, pentaerythritol tetrakis(3-mercaptobutyrate), 1,3,5-tris(3-mercaptobutyloxyethyl)-1,3,5-triazine-2,4,6(1H,3H,5H)-trione, trimethylolpropane tris(3-mercaptobutyrate), ethylene glycol bis(3-mercaptopropionate), tetraethylene glycol bis(3-mercaptopropionate), trimethylolpropane tris(3-mercaptopropionate), pentaerythritol tetrakis(3-mercaptopropionate), dipentaerythritol hexakis(3-mercaptopropionate), or tris[(3-mercaptopropionyloxy)ethyl] isocyanurate, more preferably at least one of 1,4-bis(3-mercaptobutyryloxy)butane, pentaerythritol tetrakis(3-mercaptobutyrate), 1,3,5-tris(3-mercaptobutyloxyethyl)-1,3,5-triazine-2,4,6(1H,3H,5H)-trione, or trimethylolpropane tris(3-mercaptobutyrate).

The molecular weight of the thiol compound is not particularly limited, but is, from the viewpoint of handleability in which, for example, it is easily mixed with the epoxy resin serving as Component (A) of an adhesive according to the present invention and is less likely to separate again, and a mixture obtained by mixing Component (A) and Component (B) is less likely to cause sagging or unevenness, preferably 200 to 1,000, more preferably 300 to 800.

In the present invention, the thiol compound employed may be a commercially available product, and specific examples include 1,4-bis(3-mercaptobutyryloxy)butane (trade name: Karenz MT BD1, manufactured by SHOWA DENKO K. K.), pentaerythritol tetrakis(3-mercaptobutyrate) (trade name: Karenz MT PE1, manufactured by SHOWA DENKO K. K.), 1,3,5-tris(3-mercaptobutyloxyethyl)-1,3,5-triazine-2,4,6(1H,3H,5 H)-trione (trade name: Karenz MT NR1, manufactured by SHOWA DENKO K. K.), and trimethylolpropane tris(3-mercaptobutyrate) (trade name: Karenz MT TPMB, manufactured by SHOWA DENKO K. K.).

In Component (B) used in an adhesive according to the present invention, the percentage of the polyamine compound, the acid anhydride compound, and the thiol compound is preferably 80 mass % or more, more preferably 90 mass % or more. Component (B) is also preferably composed only of one or a combination of two or more selected from the group consisting of the polyamine compound, the acid anhydride compound, and the thiol compound. When an adhesive according to the present invention includes, as Component (B), a curing component other than the polyamine compound, the acid anhydride compound, and the thiol compound, as this curing component, the above-described other compounds can be employed.

In an adhesive according to the present invention, the content of Component (B) is not particularly limited, and can be appropriately adjusted in accordance with, for example, the reaction between Component (A) and Component (B).

When Component (B) is the polyamine compound, in an adhesive according to the present invention, the content of the polyamine compound can be appropriately set in consideration of active-hydrogen equivalent, for example.

For example, the content relative to 100 parts by mass of the epoxy resin serving as Component (A) can be set at 5 to 300 parts by mass, more preferably 10 to 250 parts by mass, still more preferably 15 to 220 parts by mass. The ratio of the active-hydrogen equivalent of the polyamine compound to the epoxy equivalent of the epoxy resin serving as Component (A) (active-hydrogen equivalent/epoxy equivalent) is preferably set at 0.1 to 1.5, more preferably 0.3 to 1.0, still more preferably 0.5 to 1.0.

When Component (B) is the acid anhydride compound, in an adhesive according to the present invention, the content of the acid anhydride compound relative to 100 parts by mass of the epoxy resin serving as Component (A) is preferably 60 to 120 parts by mass, more preferably 70 to 110 parts by mass, still more preferably 80 to 100 parts by mass. When such a mixing ratio is satisfied, the curing reaction and formation of the crosslinking structure are easily controlled, to provide a cured product having high durability.

When Component (B) is the thiol compound, in an adhesive according to the present invention, the content of the thiol compound relative to 100 parts by mass of the epoxy resin serving as Component (A) is preferably 10 to 120 parts by mass, more preferably 15 to 100 parts by mass, still more preferably 20 to 90 parts by mass.

Note that, when Component (B) is the thiol compound, a curing acceleration compound is also preferably used together. The curing acceleration compound is not particularly limited, and examples include amine compounds, guanidine compounds, imidazole compounds, and phosphonium compounds. In this case, in an adhesive according to the present invention, the content of the curing acceleration compound relative to 100 parts by mass of the epoxy resin serving as Component (A) is preferably 0.1 to 10 parts by mass, more preferably 0.2 to 6 parts by mass, still more preferably 0.5 to 3 parts by mass.

### (C) Curing Acceleration Component

An adhesive according to the present invention contains, as Component (C), one or two or more acidic curing acceleration components.

Such an acidic curing acceleration component may be a proton acid or a Lewis acid.

Preferred examples of the Lewis acid include BF₃ and complexes thereof, ZnC₁₂, SuC₁₄, FeCl₃, and AlC₁₃. From the viewpoint of being excellent in pot life, more preferred is a boron trifluoride monoethylamine complex, for example.

The proton acid may be an inorganic acid or an organic acid. Examples of the inorganic acid include hydrochloric acid, phosphoric acid, and sulfuric acid. From the viewpoint of compatibility with the epoxy resin serving as Component (A), phosphoric acid is preferred. Examples of the organic acid include various compounds having an acidic proton, such as carboxylic acid compounds, sulfonic acid compounds, sulfinic acid compounds, thiophenolic compounds, phenolic compounds, and sulfonamide compounds. Of these, preferred are carboxylic acid compounds and phenolic compounds, and more preferred are phenolic compounds.

The phenolic compounds mean compounds that have a phenolic hydroxy group. Note that the phenolic compounds do not have a secondary or tertiary amine structure. In the case of having the secondary amine or tertiary amine structure, in the curing reaction of the epoxy resin, the secondary amine and tertiary amine in the phenolic compound function as an anionic polymerization catalyst for the epoxy resin, to cause unintended self-polymerization of the epoxy resin. As a result, the resultant cured product has an excessively high proportion of the self-polymerization product of the epoxy resin itself, so that the cured product has lower gas barrier performance.

The phenolic compounds may be monofunctional compounds or bi- or higher functional, polyfunctional compounds. The phenolic compounds may be low-molecular-weight compounds or high-molecular-weight compounds.

Such a phenolic compound may have, on the benzene ring forming the phenol, a substituent; examples of the substituent include saturated or unsaturated aliphatic hydrocarbon groups, aromatic hydrocarbon groups, heterocyclic groups, aromatic heterocyclic groups, a cyano group, and a nitro group. Such a saturated or unsaturated aliphatic hydrocarbon group may have, in a carbon-carbon bond, an oxygen atom (ether bond).

The phenolic compound preferably has, as a substituent, a saturated aliphatic hydrocarbon group from the viewpoint of further improving the sterilization-gas durability. This is inferentially achieved because such a phenolic compound having, as a substituent, a saturated aliphatic hydrocarbon group has improved compatibility with the epoxy resin, which further accelerates the curing reaction.

Preferred examples of the phenolic compounds include compounds represented by Formula (1) below and polymers having a constitutional component represented by at least one of Formula (2) or Formula (3) below.

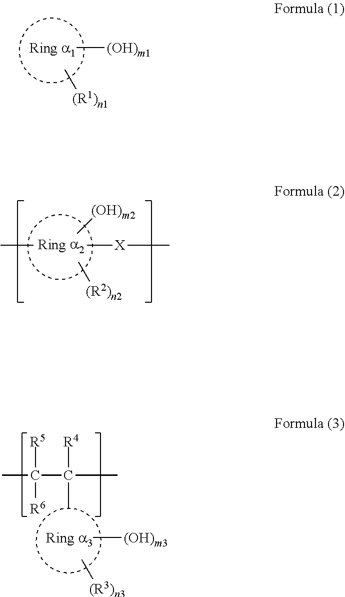

In these Formulas, R¹ to R³ represent a substituent; R⁴ to R⁶ represent a hydrogen atom or a substituent; X represents a single bond or a divalent linking group; Ring αₜ to Ring α₃ represent an aromatic hydrocarbon ring; m1 to m3 are an integer of 1 or more; and n1 to n3 are an integer of 0 or 1 or more. Note that the upper limits of m1+n1, m2+n2, and m3+n3 are respectively the upper limits of the numbers of substituents attachable to Ring α₁, Ring α₂, and Ring α₃.

Examples of the substituents in R¹ to R³ include substituents selected from Substituent group T described later; preferred examples include alkyl groups, alkenyl groups, aryl groups, heterocyclic groups, alkoxy groups, acyl groups, alkoxycarbonyl groups, alkenyloxycarbonyl groups, aryloxycarbonyl groups, alkylsulfonyl groups, arylsulfonyl groups, a cyano group, a nitro group, and —Si(R⁷)₃. R⁷'s represent an alkyl group, an alkenyl group, an aryl group, an alkoxy group, an alkenyloxy group, or an aryloxy group. These three R⁷'s may be the same or different.

These groups usable as R¹ to R³, the alkyl groups, alkenyl groups, aryl groups, heterocyclic groups, alkoxy groups, acyl groups, alkyloxycarbonyl groups, alkenyloxycarbonyl groups, aryloxycarbonyl groups, alkylsulfonyl groups, and arylsulfonyl groups have the same definitions and preferred examples as in the corresponding groups in Substituent group T described later.

These groups usable as R¹ to R³ may be unsubstituted groups or groups having a substituent. Such substituents optionally present in the groups usable as R¹ to R³ are not particularly limited, and are preferably selected from Substituent group T described later, more preferably a halogen atom or a hydroxy group. The number of the substituents is not particularly limited as long as it is one or more, and can be four or less, for example.

When n1 to n3 are an integer of 2 or more and the plurality of each of R¹ to R³ are positioned adjacent to each other such that they can be bonded together, these substituents may be bonded together to form any one of five- to seven-membered rings, a Spiro ring, or a bicyclo ring.

The groups usable as R⁷, the alkyl group, alkenyl group, aryl group, alkoxy group, alkenyloxy group, and aryloxy group have the same definitions and preferred examples as in the corresponding groups in Substituent group T described later.

These groups usable as R⁷ may be unsubstituted groups or groups having a substituent. Such substituents optionally present in the groups usable as R⁷ are not particularly limited, and are preferably selected from Substituent group T described later. The number of substituents is not particularly limited as long as it is one or more, and can be four or less, for example.

R¹ to R³ are preferably alkyl groups, alkoxy groups, alkoxycarbonyl groups, cyano groups, or nitro groups.

Examples of the substituents in R⁴ to R⁶ include substituents selected from Substituent group T described later, and preferred examples include alkyl groups, alkenyl groups, aryl groups, heterocyclic groups, alkoxy groups, alkoxycarbonyl groups, amino groups, acylamino groups, and cyano groups.

The groups usable as R⁴ to R⁶, the alkyl groups, alkenyl groups, aryl groups, heterocyclic groups, alkoxy groups, alkoxycarbonyl groups, amino groups, acylamino groups, and cyano groups have the same definitions and preferred examples as in the corresponding groups in Substituent group T described later.

These groups usable as R⁴ to R⁶ may be unsubstituted groups or groups having a substituent. Such substituents optionally present in the groups usable as R⁴ to R⁶ are not particularly limited, and are preferably selected from Substituent group T described later. The number of substituents is not particularly limited as long as it is one or more, and can be four or less, for example.

R⁴ to R⁶ are preferably hydrogen atoms, alkyl groups, alkenyl groups, aryl groups, heterocyclic groups, alkoxy groups, alkoxycarbonyl groups, amino groups, acylamino groups, or cyano groups.

The divalent linking group in X is preferably an alkylene group, an alkenylene group, an arylene group, or a heterocyclic group.

The groups usable as X, the alkylene group, alkenylene group, arylene group, and heterocyclic group respectively have the same definitions and preferred examples as in groups provided by removing one hydrogen atom from each of alkyl groups, alkenyl groups, aryl groups, and heterocyclic groups selected from Substituent group T described later.

These groups usable as X may be unsubstituted groups or groups having a substituent. Such substituents optionally present in the groups usable as X are not particularly limited, preferably selected from Substituent group T described later, and the number of substituents is not particularly limited as long as it is one or more, and can be four or less, for example.

In particular, in each of the groups usable as X, the number of carbon atoms constituting the linking moiety, specifically, the minimum number of carbon atoms linking together Ring α₂ and the point of attachment of the constitutional unit is preferably 1 to 50, more preferably 1 to 20, still more preferably 1 to 6.

X is preferably a single bond, an alkylene group, an alkenylene group, an arylene group, or a heterocyclic group, more preferably a single bond or an alkylene group.

In aromatic hydrocarbon rings in Ring α₁ to Ring α₃, the number of atoms constituting such a ring is preferably 6 to 20, more preferably 6 to 15, still more preferably 6 to 10. Specific examples include a benzene ring and a naphthalene ring.

The upper limits of m1 to m3 and the upper limits of n1 to n3 are not particularly limited as long as the totals are the upper limits or less of the number of substituents attachable to Ring α₁ to Ring α₃. Preferably, m1 to m3 are preferably 1 to 6, more preferably 1 to 3, still more preferably 1 to 2. n1 to n3 are preferably 0 to 6, more preferably 0 to 3, still more preferably 0 to 2.

### Substituent Group T

In the present invention, preferred examples of the substituents include substituents selected from Substituent group T below.

In this Specification, in the case of a mention of just a substituent, it refers to this Substituent group T; in the case of a mention of just specific groups such as an alkyl group, its corresponding groups in this Substituent group T are preferably applied.

Furthermore, in this Specification, in the case of describing alkyl groups as being different from cyclic (cyclo) alkyl groups, the alkyl groups are used with meanings including linear alkyl groups and branched alkyl groups. On the other hand, in the case of describing alkyl groups as being not different from cyclic alkyl groups and in the case of not being otherwise specified, the alkyl groups are used with meanings including linear alkyl groups, branched alkyl groups, and cycloalkyl groups. The same applies to groups (such as alkoxy groups, alkylthio groups, and alkenyloxy groups) including groups that may have ring structures (such as alkyl groups, alkenyl groups, and alkynyl groups), and compounds including groups that may have ring structures. When a group can form a cyclic skeleton, the lower limit of the number of atoms of the group forming the cyclic skeleton is three or more, preferably five or more, irrespective of the specific lower limit (described below) of the number of atoms for the group that may have the structure.

In the following description of Substituent group T, in order to distinguish, from each other, a linear or branched structure group and a cyclic structure group such as an alkyl group and a cycloalkyl group, these groups may be described separately.

The groups included in Substituent group T include the following groups:

alkyl groups (having preferably 1 to 30 carbon atoms, more preferably 1 to 20 carbon atoms, still more preferably 1 to 12 carbon atoms, still more preferably 1 to 8 carbon atoms, still more preferably 1 to 6 carbon atoms, particularly preferably 1 to 3 carbon atoms), alkenyl groups (having preferably 2 to 30 carbon atoms, more preferably 2 to 20 carbon atoms, still more preferably 2 to 12 carbon atoms, still more preferably 2 to 6 carbon atoms, still more preferably 2 to 4 carbon atoms), alkynyl groups (having preferably 2 to 30 carbon atoms, more preferably 2 to 20 carbon atoms, still more preferably 2 to 12 carbon atoms, still more preferably 2 to 6 carbon atoms, still more preferably 2 to 4 carbon atoms), cycloalkyl groups (having preferably 3 to 20 carbon atoms), cycloalkenyl groups (having preferably 5 to 20 carbon atoms), aryl groups (may be monocyclic groups or condensed ring groups (preferably condensed ring groups constituted by two to six rings); in the case of condensed ring groups, they are constituted by five- to seven-membered rings, for example; aryl groups have preferably 6 to 40 carbon atoms, more preferably 6 to 30 carbon atoms, still more preferably 6 to 26 carbon atoms, particularly preferably 6 to 10 carbon atoms), heterocyclic groups (having, as an atom forming such a ring, at least one of a nitrogen atom, an oxygen atom, a sulfur atom, a phosphorus atom, a silicon atom, or a selenium atom, may be monocyclic groups or condensed ring groups (preferably condensed ring groups constituted by two to six rings); in the case of monocyclic groups, the number of members of such a ring is preferably five to seven, more preferably five or six; the heterocyclic groups preferably have 2 to 40 carbon atoms, more preferably 2 to 20 carbon atoms; the heterocyclic groups include aromatic heterocyclic groups (heteroaryl groups) and aliphatic heterocyclic groups (aliphatic heterocycle groups)), alkoxy groups (having preferably 1 to 20 carbon atoms, more preferably 1 to 12 carbon atoms), alkenyloxy groups (having preferably 2 to 20 carbon atoms, more preferably 2 to 12 carbon atoms), alkynyloxy groups (having preferably 2 to 20 carbon atoms, more preferably 2 to 12 carbon atoms), cycloalkyloxy groups (having preferably 3 to 20 carbon atoms), aryloxy groups (having preferably 6 to 40 carbon atoms, more preferably 6 to 26 carbon atoms, still more preferably 6 to 14 carbon atoms), heterocyclic oxy groups (having preferably 2 to 20 carbon atoms),

alkoxycarbonyl groups (having preferably 2 to 20 carbon atoms), cycloalkoxycarbonyl groups (having preferably 4 to 20 carbon atoms), aryloxycarbonyl groups (having preferably 6 to 20 carbon atoms), amino groups (having preferably 0 to 20 carbon atoms; including the unsubstituted amino group (—NH₂), (mono- or di-)alkylamino groups, (mono- or di-)alkenylamino groups, (mono- or di-)alkynylamino groups, (mono- or di-)cycloalkylamino groups, (mono- or di-)cycloalkenylamino groups, (mono- or di-)arylamino groups, and (mono-or di-)heterocyclic amino groups; such groups serving as substituents for the unsubstituted amino group have the same definitions as in the corresponding groups in Substituent group T), sulfamoyl groups (having preferably 0 to 20 carbon atoms; preferably alkyl, cycloalkyl, or aryl sulfamoyl groups), acyl groups (having preferably 1 to 20 carbon atoms, more preferably 2 to 15 carbon atoms), acyloxy groups (having preferably 1 to 20 carbon atoms), carbamoyl groups (having preferably 1 to 20 carbon atoms; preferably alkyl, cycloalkyl, or aryl carbamoyl groups),

acylamino groups (having preferably 1 to 20 carbon atoms), sulfonamide groups (having preferably 0 to 20 carbon atoms; preferably alkyl, cycloalkyl, or aryl sulfonamide groups), alkylthio groups (having preferably 1 to 20 carbon atoms, more preferably 1 to 12 carbon atoms), cycloalkylthio groups (having preferably 3 to 20 carbon atoms), arylthio groups (having preferably 6 to 40 carbon atoms, more preferably 6 to 26 carbon atoms, still more preferably 6 to 14 carbon atoms), heterocyclic thio groups (having preferably 2 to 20 carbon atoms), alkyl, cycloalkyl, or aryl sulfonyl groups (having preferably 1 to 20 carbon atoms),

silyl groups (having preferably 1 to 30 carbon atoms, more preferably 1 to 20 carbon atoms; preferably alkyl-, aryl-, alkoxy-, or aryloxy-substituted silyl groups), silyloxy groups (having preferably 1 to 20 carbon atoms; preferably alkyl-, aryl-, alkoxy-, or aryloxy-substituted silyloxy groups), a hydroxy group, a cyano group, a nitro group, halogen atoms (such as a fluorine atom, a chlorine atom, a bromine atom, or an iodine atom), an oxygen atom (specifically, >CH₂ forming the ring is replaced by >C═O), a carboxy group (—CO₂H), a phosphono group [—PO(OH)₂], a phosphoryl group [—O—PO(OH)₂], a sulfo group (—SO₃H), a borate group [—B(OH)₂], onio groups (including ammonio groups including cyclic ammonio, a sulfonio group, and a phosphonio group; having preferably 0 to 30 carbon atoms, more preferably 1 to 20 carbon atoms), a sulfanyl group (—SH), an amino acid residue, and a polyamino acid residue.

In addition, included are such alkyl groups, alkenyl groups, alkynyl groups, cycloalkyl groups, cycloalkenyl groups, aryl groups, heterocyclic groups, alkoxy groups, alkenyloxy groups, alkynyloxy groups, cycloalkyloxy groups, aryloxy groups, heterocyclic oxy groups, alkoxycarbonyl groups, cycloalkoxycarbonyl groups, aryloxycarbonyl groups, amino groups, sulfamoyl groups, acyl groups, acyloxy groups, carbamoyl groups, acylamino groups, sulfonamide groups, alkylthio groups, cycloalkylthio groups, arylthio groups, heterocyclic thio groups, and alkyl, cycloalkyl, or aryl sulfonyl groups that have, as substituents, carboxy groups, phosphono groups, sulfo groups, onio groups, amino acid residues, or polyamino acid residues.

The substituents selected from Substituent group T are more preferably alkyl groups, alkenyl groups, cycloalkyl groups, aryl groups, heterocyclic groups, alkoxy groups, cycloalkoxy groups, aryloxy groups, alkoxycarbonyl groups, cycloalkoxycarbonyl groups, amino groups, acylamino groups, cyano groups, or halogen atoms, and are particularly preferably alkyl groups, alkenyl groups, aryl groups, heterocyclic groups, alkoxy groups, alkoxycarbonyl groups, amino groups, acylamino groups, or cyano groups.

The substituents selected from Substituent group T also include, unless otherwise specified, groups that are combinations of a plurality of groups among the above-described groups. For example, when a compound or substituents etc. include an alkyl group, an alkenyl group, or the like, these may be substituted or may not be substituted. In the case of including an aryl group, a heterocyclic group, and the like, these may be monocyclic groups or condensed rings, and may be substituted or may not be substituted.

In the polymer having a constitutional component represented by at least one of Formula (2) above or Formula (3) above, the content of the constitutional component represented by at least one of Formula (2) above or Formula (3) above is not particularly limited; however, for example, the content relative to all the constitutional components constituting the polymer is preferably 70 mass % or more, more preferably 80 mass % or more, still more preferably 90 mass % or more.

Another constitutional component of the polymer other than Formula (2) above and Formula (3) above is not particularly limited.

The phenolic compound preferably has a molecular weight of 90 or more and less than 5000, more preferably 90 or more and 3000 or less, still more preferably 90 or more and 25000 or less. The molecular weight can be set in such a range to thereby lower the probability of flowing out of the phenolic compound from the cured product, and to provide sufficient solubility in the epoxy resin serving as Component (A).

The phenolic compound preferably has a phenolic-hydroxy-group equivalent of 100 to 400, more preferably 100 to 300. The phenolic-hydroxy-group equivalent can be set in such a range, so that the polarity does not become excessively high, sufficient solubility in the epoxy resin serving as Component (A) is provided, and a sufficient acceleration effect is exerted. The phenolic-hydroxy-group equivalent is a value obtained by dividing the molecular weight of the phenolic compound by the number of moles of the phenolic hydroxy groups of the phenolic compound.

The phenolic hydroxy group preferably has a pKa value of 8 to 12, more preferably 9 to 11. The pKa value can be set in such a range to thereby suppress a side reaction that is cationic polymerization of the epoxy resin and to exert a sufficient acceleration effect.

This pKa value of the phenolic compound is a value calculated using ChemDraw Professional 16.0 (manufactured by PerkinElmer, Inc.). When the phenolic compound is a polymer, in a constitutional unit having a phenolic hydroxy group (preferably a constitutional unit represented by Formula (2) or (3) above), direct bonds are regarded as hydrogen atoms, and the pKa value of the resultant compound is calculated as the pKa value of the polymer.

Incidentally, when the phenolic compound has two or more phenolic hydroxy groups having different pKa values, the above-described pKa value range is satisfied when at least one phenolic hydroxy group has a pKa value within the above-described pKa range.

The following are preferred specific examples of the phenolic compound usable for the present invention. The numbers at the parentheses are the repeating numbers of the constitutional units within the parentheses.

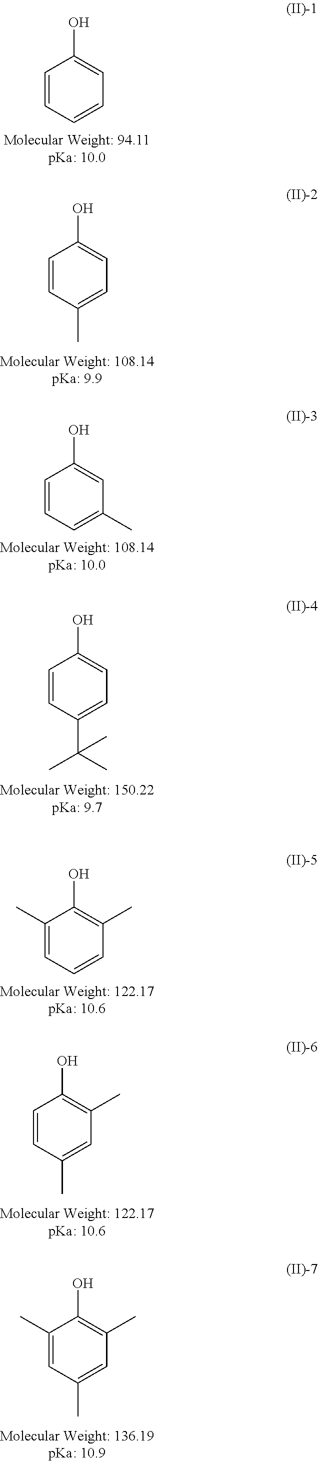

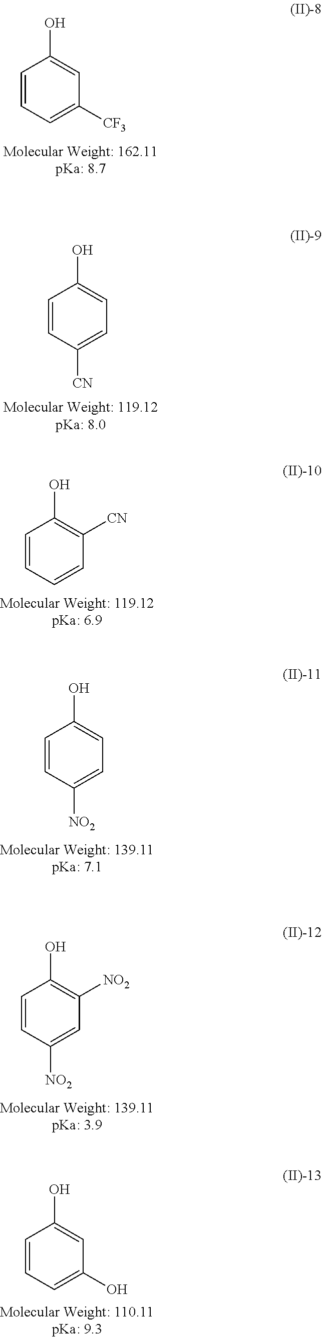 [SMILES: Oc1cccc(C(F)(F)F)c1 ; N#Cc1ccc(O)cc1 ; N#Cc1ccccc1O ; O=[N+]([O-])c1ccc(O)cc1 ; O=[N+]([O-])c1ccc(O)c([N+](=O)[O-])c1 ; Oc1cccc(O)c1]

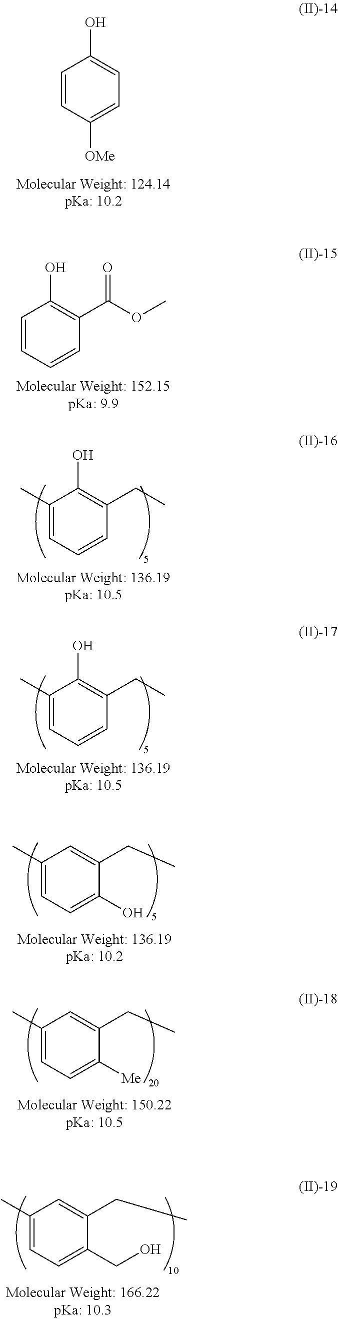

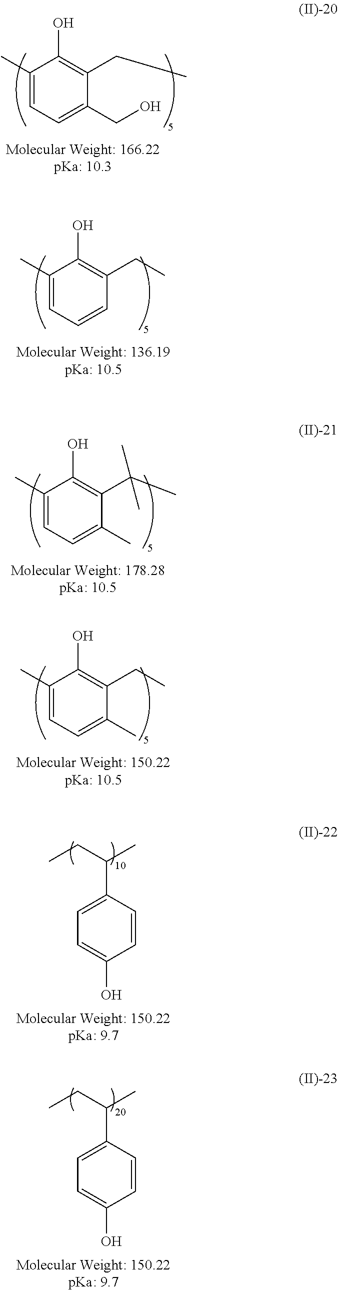

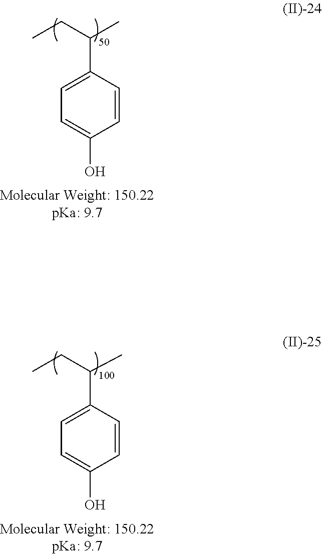

These phenolic compounds can be synthesized in the standard manner; alternatively, commercially available products may be used.

The acidic curing acceleration components other than the phenolic compounds can also be synthesized in the standard manner; alternatively, commercially available products may be used.

In an adhesive according to the present invention, the formulation amount of the curing acceleration component relative to 100 parts by mass of the epoxy resin is preferably 3 to 20 parts by mass, more preferably 5 to 15 parts by mass, still more preferably 5 to 10 parts by mass. When the formulation amount is set in such a range, a sufficient curing acceleration effect can be exerted without degradation of properties of the cured product.

In an adhesive according to the present invention, the formulation amount ratio of Curing component (B) and Curing acceleration component (C) in parts by mass is preferably Curing component (B):Curing acceleration component (C)=30:1 to 1.5:1, more preferably Curing component (B):Curing acceleration component (C)=10:1 to 2:1, still more preferably Curing component (B):Curing acceleration component (C)=8:1 to 4:1.

### Cured Product

A cured product according to the present invention is a cured product generated by curing an adhesive according to the present invention. Specifically, a cured product according to the present invention is used as a member forming a bonding portion in an endoscope. The curing temperature of an adhesive according to the present invention is not particularly limited, and can be appropriately set in accordance with Component (B) contained in an adhesive according to the present invention. The components can be mixed together in the standard manner. This mixing is preferably performed while bubbles are removed, and hence is ordinarily performed under reduced pressure.

Specifically, in the case of containing, as Component (B), a polyamine compound, in an adhesive according to the present invention, the curing reaction efficiently proceeds even in a low-temperature range to provide a cured product according to the present invention. The curing temperature is, for example, preferably 100° C. or less, more preferably 80° C. or less, still more preferably 60° C. or less, particularly preferably 50° C. or less; curing can be achieved even at room temperature (25° C.) or less, which is preferred. In order to sufficiently cause the curing reaction, the curing temperature is preferably 0° C. or more, more preferably 10° C. or more. The curing-reaction time can be appropriately set in accordance with the purpose. Ordinarily, the curing reaction is caused for 1.5 to 200 hours, to provide the cured product.

In the case of containing, as Component (B), an acid anhydride compound, for example, heating at 25 to 220° C. for 0.5 to 48 hours provides a cured product according to the present invention. The curing temperature is preferably 200° C. or less, more preferably 180° C. or less. In order to sufficiently cause the curing reaction, the curing temperature is preferably 120° C. or more, more preferably 140° C. or more.

In the case of containing, as Component (B), a thiol compound, for example, heating at −20 to 150° C. for 10 minutes to 72 hours achieves curing to thereby provide a cured product according to the present invention. The curing temperature is preferably 100° C. or less, more preferably 80° C. or less. In order to sufficiently cause the curing reaction, the curing temperature is preferably 0° C. or more, more preferably 10° C. or more.

Note that, in the case of containing, as Component (C), a phenolic compound, the curing temperature is preferably 15° C. to 100° C., more preferably 20° C. to 80° C. Such a range can be satisfied, to thereby sufficiently exert the acceleration effect, and to suppress the reaction between the phenolic compound and the epoxy resin.

Note that the curing temperature of an adhesive according to the present invention is preferably set as low as possible from the viewpoint of reducing repeated exposure of the endoscope to high temperatures during the production steps.

### Endoscope

In an endoscope according to the present invention, a cured product according to the present invention is used to fix a constituent member. This phrase “a cured product according to the present invention is used to fix a constituent member” means that at least one of the constituent members of the endoscope is fixed to a support member using a cured product according to the present invention.

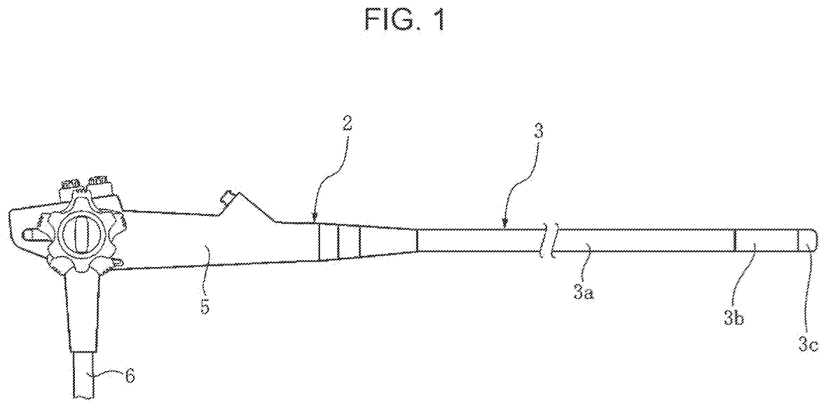

An example of an endoscope (electronic endoscope) according to the present invention will be described. The electronic endoscope includes therein an endoscopic flexible tube (hereafter, the endoscopic flexible tube may be simply referred to as “flexible tube”), and is widely used as a medical device. In the example illustrated in FIG. 1, an electronic endoscope 2 includes an insertion section 3 to be inserted into the body cavity, a main-body operation section 5 coupled to the base-end portion of the insertion section 3, and a universal cord 6 connected to a processor device and a light source device. The insertion section 3 is constituted by a flexible tube 3a coupled to the main-body operation section 5, an angle portion 3b coupled to the flexible tube 3a, and a distal-end portion 3c coupled to the distal end of the angle portion 3b and mainly constituted by metal (such as stainless steel) members. This distal-end portion 3c houses an imaging device (not shown) for imaging the inside of the body cavity. The flexible tube 3a, which accounts for most of the length of the insertion section 3, has flexibility substantially over the whole length; in particular, the portion inserted into inner regions such as the body cavity has a more flexible structure.

In FIG. 1, from the main-body operation section 5 to the distal-end surface of the distal-end portion 3c, a plurality of channels (tubes, not shown) extending throughout in the axial direction of the insertion section 3 are formed.

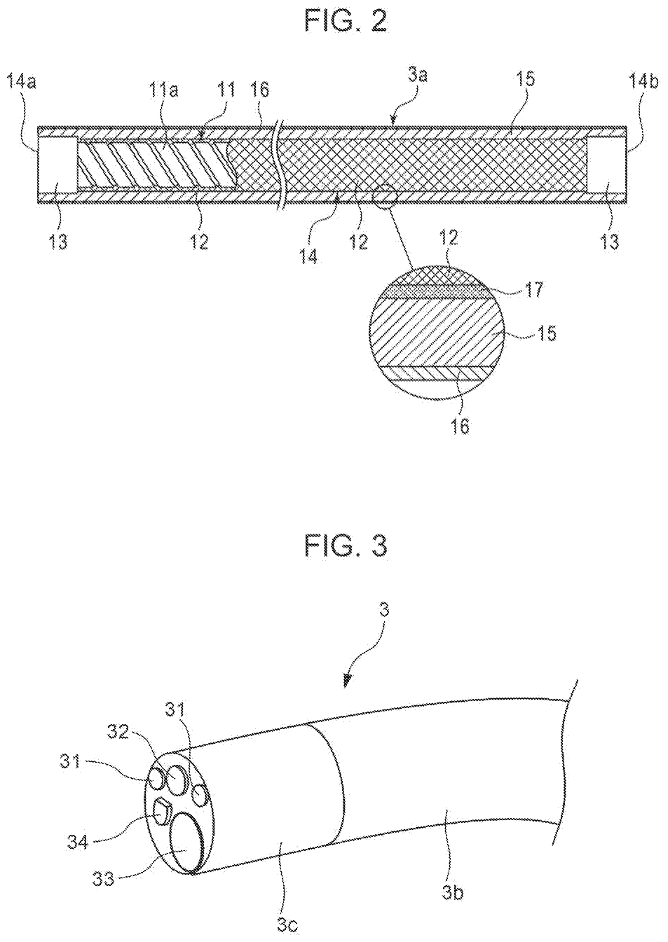

The flexible tube 3a in FIG. 1 has a configuration in which, as illustrated in FIG. 2, the outer peripheral surface of a flexible-tube base 14 is covered with a resin layer 15.

Reference sign 14a denotes the distal-end side (distal-end portion 3c side) while Reference sign 14b denotes the base-end side (main-body operation section 5 side).

The flexible-tube base 14 is formed by covering a spiral tube 11, which is disposed on the innermost side and formed by spirally winding a metal strip 11a, with a sleeve mesh body 12 formed by knitting metal wires. Both ends of the flexible-tube base 14 are fitted with metal caps 13. This resin layer 15 is bonded to the flexible-tube base 14 with an adhesive-cured-product layer 17 therebetween. This adhesive-cured-product layer 17 can be formed by applying and curing an adhesive according to the present invention. The adhesive-cured-product layer (bonding portion) 17 is drawn as a uniform and thick layer for the purpose of illustration, but does not necessarily have this form, and may be disposed in an amorphous form between the resin layer 15 and the flexible-tube base 14. Alternatively, the layer may barely have a thickness and the resin layer 15 and the flexible-tube base 14 may be bonded together substantially in contact with each other.

The outer surface of the resin layer 15 is coated with a coating layer 16 having chemical resistance and containing, for example, fluorine. Incidentally, the adhesive-cured-product layer 17, the resin layer 15, and the coating layer 16 are drawn at large thicknesses relative to the diameter of the flexible-tube base 14 for the purpose of clearly illustrating the layer structure.

As illustrated in FIG. 3, at the distal-end surface of the distal-end portion 3c, illumination windows 31, an observation window 32, and a forceps port 33 are formed. In addition, in order to wash the distal-end surface as needed, a nozzle 34 for sending out water and the air is formed. The illumination windows 31, the observation window 32, the forceps port 33, and the nozzle 34 extend through channels and coupled to the main-body operation section 5.

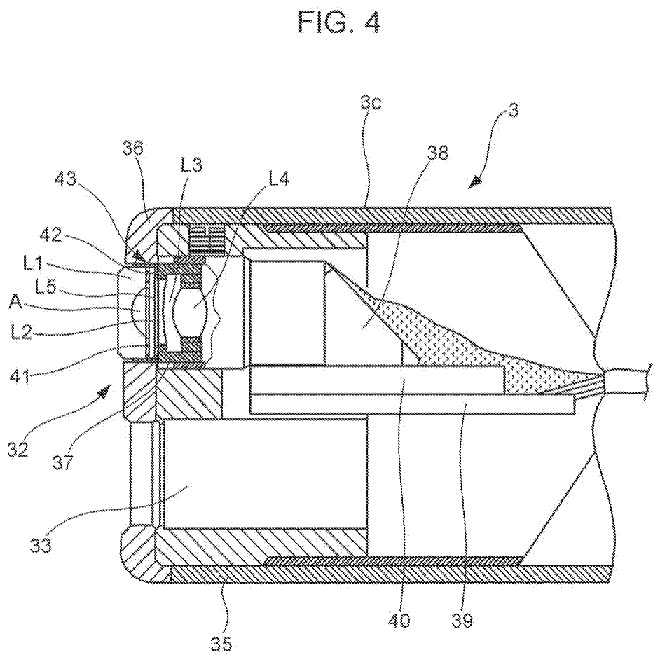

As illustrated in FIG. 4, the distal-end portion 3c is constituted by a distal-end-portion main body 35 formed of metal, and a distal-end cap 36 formed of an electrically insulating member.

At the observation window 32, an observation unit 43, which is an optical device, is disposed. In the observation unit 43, within a lens holder 37, an objective optical system constituted by lenses L1 to L5 is fixed using adhesive cured products 41 and 42. These adhesive cured products 41 and 42 are formed by applying and curing an adhesive according to the present invention. In this objective optical system, Reference sign A denotes an air layer. To an end surface of the lens holder 37, a prism 38 is bonded and fixed. This prism 38 perpendicularly deflects the optical axis of the objective optical system. This prism 38 is fixed to a solid image pickup element 40. The solid image pickup element 40 is fixed to a substrate 39. These can be fixed also with an adhesive according to the present invention.

### Method for Producing Endoscope

A method for producing an endoscope according to the present invention is not particularly limited as long as it includes using an adhesive according to the present invention to fix an endoscope constituent member; as the steps other than the fixing of the endoscope constituent member, ordinary production steps can be employed to produce an endoscope according to the present invention.

The endoscope constituent member to be fixed is not particularly limited in terms of material, and examples include resin members, metal members, and glass members. The endoscope constituent member can be fixed to a support member or the like of an endoscope in the following manner: for example, the components included in an adhesive according to the present invention are mixed together preferably under a reduced pressure; subsequently, the mixture is injected or applied to the application point, and heated, for example, at −10 to 60° C. (preferably 0 to 60° C., more preferably 10 to 50° C.) for 1.5 to 200 hours.

Hereinafter, usage forms of the adhesive in a method for producing an endoscope according to the present invention will be described with reference to specific examples; however, the present invention is not limited to these.

Of endoscope constituent members fixed using an adhesive according to the present invention, a resin member is, for example, a tube inserted through the insertion section of the endoscope. Examples of the resin material forming the tube include fluororesins such as TEFLON (registered trademark), polysulfone, polyesters, polyolefins, and silicone. An adhesive according to the present invention can be used for, for example, bonding between a metal member or a glass member of the insertion section of the endoscope and the tube (fixing of the metal member or the glass member to the tube).

In addition, as described above, the adhesive can also be used to form the adhesive-cured-product layer 17 in FIG. 2. In addition, the adhesive can also be used to bond together, in FIG. 2, the resin layer 15 and the coating layer 16.

An adhesive according to the present invention can be used for outer-surface finishing and fixing of an end portion (distal-end side (angle portion 3b side) end portion of the flexible tube 3a) of the flexible outer cover tube (resin layer 15). Specifically, the end portion of the resin layer 15 of the flexible tube 3a is externally bound tightly using a thread and fixed to the internal member, and subsequently the adhesive is applied so as to cover the thread and cured. The outermost layer of the distal-end-side end portion of the flexible tube 3a is thus formed from an adhesive according to the present invention, so that the thread in this distal-end-side end portion becomes less likely to fray, and the insertion section is easily inserted into the body cavity.

In addition, an adhesive according to the present invention can be used for bonding between the distal-end portion 3c and the angle portion 3b and/or bonding between the insertion section 3 and the main-body operation section 5. For example, the distal-end portion 3c and the angle portion 3b are bonded together using an adhesive according to the present invention; subsequently, the region at and near the bonding portion between the distal-end portion 3c and the angle portion 3b is bound tightly using a thread to reinforce the bonding, and the adhesive is applied so as to cover the thread, and cured. The same applies to bonding between the insertion section 3 and the main-body operation section 5.

In addition, an adhesive according to the present invention can also be used for fixing of various tubes inserted through the insertion section of the endoscope, onto the distal-end portion 3c and/or the main-body operation section 5.

In addition, an adhesive according to the present invention is also preferably used, in the distal-end portion 3c, for sealing of the illumination windows 31 and the observation window 32 (fixing of the glass members). The adhesive can be applied at large thicknesses, to thereby smooth the peripheral corners of the lenses, and to block lateral entry of light into the lenses.

In addition, an adhesive according to the present invention can be used for fixing of members such as assembly of the imaging device housed within the distal-end portion 3c, bonding of parts, and sealing of the solid image pickup element 40. The imaging device has an optical system constituted by a plurality of optical parts such as the lenses L1 to L5 and the prism 38, and the solid image pickup element 40 that photoelectrically converts optical images formed by the optical system into imaging signals, such as a CCD (Charge Coupled Device). An adhesive according to the present invention can be used for, for example, bonding together of optical parts formed of a material such as glass that are the lenses L1 to L5, the prism 38, and the like, and bonding of the lenses L1 to L5, the prism 38, and the like to the substrate 39 formed of resin or metal; this bonding achieves fixing of the glass members, and fixing of the metal members.

In addition, an adhesive according to the present invention can be used for bond-fixing and sealing between the solid image pickup element 40 and the substrate 39. This bonding achieves fixing of the metal members constituting the solid image pickup element, the substrate, and the like.

Thus, a method for producing an endoscope according to the present invention includes a step of using an adhesive according to the present invention to fix an endoscope constituent member.

### EXAMPLES

The present invention will be described further in detail with reference to Examples; however, the present invention is not construed as being limited to Examples below. In Examples below, “room temperature” means 25° C. The formulation amount of each component means the formulation amount of the component itself. Specifically, when the raw material includes a solvent, the formulation amount does not include the amount of the solvent.

### Preparation Example: Preparation of Adhesives

Epoxy resins, curing components, and curing acceleration components in Tables below were weighed so as to satisfy the formulation ratios in Tables below, and mixed.

The resultant mixtures were, under stirring with an “AWATORI-RENTARO ARV-310 (trade name, manufactured by THINKY CORPORATION)” at room temperature, at a reduced pressure of 1.0 Pa, and at a revolution rate of 2000 rpm, defoamed for 5 minutes, to provide adhesives. The adhesives were cured with a MINI TEST PRESS (manufactured by Toyo Seiki Seisaku-sho, Ltd.) at 80° C. for 6 hours, to provide sheet-shaped cured products having a length of 100 mm, a width of 100 mm, and a thickness of 0.4 mm.

### Test Examples

### EOG Barrier Performance

As a water-vapor transmission cup satisfying the conditions defined in JIS Z 0208, a screw-fastener-type water-vapor transmission cup (manufactured by Imoto machinery Co., LTD., material: stainless steel, area of transmission: 60 mmΦ) was prepared; into the water-vapor transmission cup at its bottom, a PROSHARE EOG sterilization indicator tape (trade name, manufactured by AS ONE Corporation) was placed; from the sheet-shaped cured product prepared above, a circular test piece (70 mmΦ) was obtained and used, and the screws were fastened to seal the water-vapor transmission cup.

The sealed water-vapor transmission cup was subjected to, using an ethylene oxide gas sterilizer EQ-70 (trade name, manufactured by MIURA CO., LTD.), an ethylene oxide gas (EOG) sterilization treatment of a high-temperature sterilization course at 55° C. for 210 hours. After the EOG sterilization treatment, the tint of the indicator tape was visually inspected. The indicator tape, upon exposure to EOG, gradually changes from green to brown.

The change in the tint of the indicator tape before and after the sterilization treatment was graded in accordance with the following grades to perform evaluation of EOG barrier performance. Grades “S” to “B” are pass grades of this test.

### Evaluation Grades

S: Even after the EOG sterilization treatment was performed for 210 hours, the color of the indicator tape did not change and remained green.

A: After the EOG sterilization treatment was performed for 210 hours, the color of the indicator tape changed slightly to the brown side (an intermediate position between green and brown), compared with before the sterilization treatment.

B: The EOG sterilization treatment time at which the color of the indicator tape completely changed to brown was 120 hours or more and less than 210 hours.

C: The EOG sterilization treatment time at which the color of the indicator tape completely changed to brown was less than 120 hours.

The results are described in Tables below.

### STERRAD Barrier Performance

As a water-vapor transmission cup satisfying the conditions defined in JIS Z 0208, a screw-fastener-type water-vapor transmission cup (manufactured by Imoto machinery Co., LTD., material: stainless steel, area of transmission: 60 mmΦ) was prepared; into the water-vapor transmission cup at its bottom, a PROSHARE EOG sterilization indicator tape (trade name, manufactured by AS ONE Corporation) was placed; from the sheet-shaped cured product prepared above, a circular test piece (70 mmΦ) was obtained and used, and the screws were fastened to seal the water-vapor transmission cup.

The sealed water-vapor transmission cup was subjected to, using a STERRAD (registered trademark) NX (trade name, manufactured by Johnson & Johnson), a sterilization cycle of the advanced cycle to perform a hydrogen peroxide plasma sterilization treatment. Incidentally, this sterilization treatment was defined as 1 cycle. After the STERRAD sterilization treatment, the tint of the indicator tape was visually inspected. The indicator tape upon exposure to hydrogen peroxide gradually changes from red to yellow.

The change in the tint of the indicator tape before and after the sterilization treatment was graded in accordance with the following grades to perform evaluation of STERRAD barrier performance. Grades “S” to “B” are pass grades in this test.

### Evaluation Grades

S: Even after the STERRAD sterilization treatment was performed for 100 cycles, the color of the indicator tape did not change and remained red.

A: After the STERRAD sterilization treatment was performed for 100 cycles, the color of the indicator tape changed slightly to the yellow side (an intermediate position between red and yellow), compared with before the sterilization treatment.

B: The number of cycles of the STERRAD sterilization treatment at which the color of the indicator tape completely changed to yellow was 50 to 99 cycles.

C: The number of cycles of the STERRAD sterilization treatment at which the color of the indicator tape completely changed to yellow was 1 to 49 cycles.

The results are described in Tables below.

TABLE 1-1

| | (A) Epoxy resin | | (B) Curing agent | | (C) Acidic curing acceleration agent | | | | Evaluation results | |
|---|---|---|---|---|---|---|---|---|---|---|
| | | Parts by | | Parts by | | Molecular | | Parts by | EOG barrier | STERRAD barrier |
| | Type | mass | Type | mass | Type | weight | pKa | mass | performance | performance |
| Example 1 | A-2 | 100 | B-1-6 | 40 | C-1-1 | 98 | 2.1 | 10 | B | B |
| Example 2 | A-2 | 100 | B-1-6 | 40 | C-1-2 | 60 | 4.8 | 10 | B | B |
| Example 3 | A-2 | 100 | B-1-6 | 40 | C-1-3 | 113 | None | 10 | B | B |
| Example 4 | A-2 | 100 | B-1-6 | 40 | C-2-2 | 108 | 9.9 | 10 | S | S |
| Example 5 | A-1 | 100 | B-1-6 | 40 | C-2-2 | 108 | 9.9 | 10 | A | B |
| Example 6 | A-3 | 100 | B-1-6 | 40 | C-2-2 | 108 | 9.9 | 10 | A | B |
| Example 7 | A-4 | 100 | B-1-6 | 40 | C-2-2 | 108 | 9.9 | 10 | A | B |
| Example 8 | A-5 | 100 | B-1-6 | 40 | C-2-2 | 108 | 9.9 | 10 | A | B |
| Example 9 | A-2 | 100 | B-1-6 | 40 | C-2-4 | 150 | 9.7 | 10 | S | S |
| Example 10 | A-2 | 100 | B-1-6 | 40 | C-2-12 | 139 | 3.9 | 10 | A | B |
| Example 11 | A-2 | 100 | B-1-6 | 40 | C-2-14 | 124 | 10.2 | 10 | S | S |
| Example 12 | A-2 | 100 | B-1-6 | 40 | C-2-16 | 700 | 10.4 | 10 | S | S |
| Example 13 | A-2 | 100 | B-1-6 | 40 | C-2-18 | 3000 | 10.5 | 10 | A | A |
| Example 14 | A-2 | 100 | B-1-6 | 40 | C-2-24 | 7000 | 9.8 | 10 | A | B |
| Example 15 | A-2 | 100 | B-1-6 | 40 | C-2-25 | 14000 | 9.8 | 10 | A | B |
| Example 16 | A-2 | 100 | B-1-6 | 40 | C-2-2 | 108 | 9.9 | 3 | B | B |
| Example 17 | A-2 | 100 | B-1-6 | 40 | C-2-2 | 108 | 9.9 | 5 | S | S |
| Example 18 | A-2 | 100 | B-1-7 | 40 | C-2-2 | 108 | 9.9 | 20 | B | B |
| Example 19 | A-2 | 100 | B-1-7 | 40 | C-2-2 | 108 | 9.9 | 10 | S | S |
| Example 20 | A-2 | 100 | B-1-7 | 40 | C-2-4 | 150 | 9.7 | 10 | S | S |
| Example 21 | A-2 | 100 | B-1-7 | 40 | C-2-16 | 700 | 10.4 | 10 | S | S |
| Example 22 | A-2 | 100 | B-1-7 | 40 | C-2-24 | 7000 | 9.8 | 10 | A | B |

TABLE 1-2

| | (A) Epoxy resin | | (B) Curing agent | | (C) Acidic curing acceleration agent | | | | Evaluation results | |
|---|---|---|---|---|---|---|---|---|---|---|
| | | Parts by | | Parts by | | Molecular | | Parts by | EOG barrier | STERRAD barrier |
| | Type | mass | Type | mass | Type | weight | pKa | mass | performance | performance |
| Example 23 | A-2 | 100 | B-1-7 | 40 | C-2-25 | 14000 | 9.8 | 10 | A | B |
| Example 24 | A-2 | 100 | B-1-7 | 40 | C-2-2 | 108 | 9.9 | 3 | B | B |
| Example 25 | A-2 | 100 | B-1-6 | 15 | C-2-2 | 108 | 9.9 | 10 | B | B |
| Example 26 | A-2 | 100 | B-1-6 | 100 | C-2-2 | 108 | 9.9 | 10 | B | B |
| Example 27 | A-2 | 100 | B-1-1 | 40 | C-2-2 | 108 | 9.9 | 10 | B | A |
| Example 28 | A-2 | 100 | B-1-3 | 40 | C-2-2 | 108 | 9.9 | 10 | B | A |
| Example 29 | A-2 | 100 | B-1-11 | 40 | C-2-2 | 108 | 9.9 | 10 | B | B |
| Example 30 | A-2 | 100 | B-1-12 | 40 | C-2-2 | 108 | 9.9 | 10 | B | B |
| Example 31 | A-2 | 100 | B-2-1 | 100 | C-2-2 | 108 | 9.9 | 10 | B | B |
| Example 32 | A-2 | 100 | B-2-2 | 58 | C-2-2 | 108 | 9.9 | 10 | B | B |
| Comparative Example 1 | A-2 | 100 | B-1-12 | 40 | X-1 | 135 | None | 10 | C | C |
| Comparative Example 2 | A-2 | 100 | B-1-12 | 40 | X-2 | 262 | None | 10 | C | C |
| Comparative Example 3 | A-2 | 100 | B-1-12 | 40 | None | None | None | None | C | C |
| Comparative Example 4 | A-2 | 100 | B-1-1 | 40 | None | None | None | None | C | C |
| Comparative Example 5 | A-2 | 100 | B-1-1 | 40 | X-1 | 135 | None | 10 | C | C |
| Comparative Example 6 | A-2 | 100 | B-2-1 | 100 | X-1 | 135 | None | 10 | C | C |
| Comparative Example 7 | A-2 | 100 | B-2-2 | 58 | X-2 | 262 | None | 10 | C | C |
| Comparative Example 8 | A-2 | 100 | B-1-1 | 40 | X-3 | 265 | None | 10 | C | C |

### (A) Epoxy Resin

### A-1:

Bisphenol A diglycidyl ether (trade name: “jER 825”, manufactured by Mitsubishi Chemical Corporation, epoxy equivalent: 170)

### A-2:

Bisphenol A diglycidyl ether (trade name: “jER 828”, manufactured by Mitsubishi Chemical Corporation, epoxy equivalent: 190)

### A-3:

Bisphenol A diglycidyl ether (trade name: “jER 834”, manufactured by Mitsubishi Chemical Corporation, epoxy equivalent: 230)

### A-4:

Bisphenol F diglycidyl ether (trade name: “EPICLON 830”, manufactured by DIC Corporation, epoxy equivalent: 170)

### A-5:

Epoxy novolac resin (product number: 406775, manufactured by Sigma-Aldrich Corporation, epoxy equivalent: 170)

### (B) Curing Component

### (1) Polyamine Compounds

### B-1-1:

1,6-Hexanediamine (manufactured by Tokyo Chemical Industry Co., Ltd., active-hydrogen equivalent: 29)

(B-45, the above-described specific example of polyamine compound)

### B-1-3:

Trimethylhexamethylenediamine (manufactured by Tokyo Chemical Industry Co., Ltd., active-hydrogen equivalent: 40)

(B-46, the above-described specific example of polyamine compound)

### B-1-6:

Polyoxyalkylenediamine (trade name: D400, manufactured by Mitsui Fine Chemicals, Inc., active-hydrogen equivalent: 100)

### B-1-7:

Polyoxyalkylenetriamine (trade name: T403, manufactured by Mitsui Fine Chemicals, Inc., active-hydrogen equivalent: 73)

### B-1-11:

m-Xylylenediamine (manufactured by Tokyo Chemical Industry Co., Ltd., active-hydrogen equivalent: 34)

(B-53, the above-described specific example of polyamine compound)

### B-1-12:

HV953U (trade name, manufactured by Nagase ChemteX Corporation, polyamidoamine, active-hydrogen equivalent: 120)

### (2) Acid Anhydride Compounds

### B-2-1:

4-Methylhexahydrophthalic anhydride/hexahydrophthalic anhydride=70/30 (trade name: RIKACID MH-700, manufactured by New Japan Chemical Co., Ltd.)

(AH-12 and AH-11, the above-described specific examples of acid anhydride compound)

### (3) Thiol Compound

### B-2-2:

Pentaerythritol tetrakis(3-mercaptobutyrate) (trade name: Karenz MT PE1, manufactured by SHOWA DENKO K. K.)

### (C) Curing Acceleration Component

### (1) Proton Acid (Inorganic Acid)

C-1-1: Phosphoric acid (manufactured by Wako Pure Chemical Industries, Ltd.)

### (2) Lewis acid

C-1-3: Boron trifluoride monoethylamine complex (manufactured by Wako Pure Chemical Industries, Ltd.)

### (3) Proton Acids (Organic Acids)

C-1-2: Acetic acid (manufactured by Wako Pure Chemical Industries, Ltd.)
C-2-2, C-2-4, C-2-12, C-2-14, C-2-16, C-2-18, C-2-24, and C-2-25: respectively corresponding to the above-described specific examples of the phenolic compound that are (II)-2, (II)-4, (II)-12, (II)-14, (II)-16, (II)-18, (II)-24, and (II)-25.

### Other Comparative Curing Acceleration Components

X-1: Benzyldimethylamine, manufactured by Wako Pure Chemical Industries, Ltd.
X-2: Triphenylphosphine, manufactured by Wako Pure Chemical Industries, Ltd.
X-3: 2,4,6-Tris(dimethylaminomethyl)phenol, manufactured by Wako Pure Chemical Industries, Ltd.

As described in Table above, the epoxy adhesives of Comparative Examples 3 and 4 do not contain curing acceleration components. The cured products obtained by curing the adhesives of Comparative Examples 3 and 4 were poor in both of EOG barrier performance and STERRAD barrier performance.

The epoxy adhesives of Comparative Examples 1, 2 and 5 to 8 contain, as curing acceleration components, benzylmethylamine, triphenylphosphine, or 2,4,6-tris(dimethylaminomethyl)phenol. The cured products obtained by curing the adhesives of Comparative Examples 1, 2, and 5 to 8 were also poor in both of EOG barrier performance and STERRAD barrier performance.

By contrast, for the epoxy adhesives of Examples 1 to 32 containing acidic curing acceleration components according to the present invention, the cured products obtained by curing these were found to have sufficient EOG barrier performance and STERRAD barrier performance.

Among these Examples, of the cured products obtained from the adhesives of Examples 1 to 4 and 9 to 15, which have the same conditions except for the type of the acidic curing acceleration component, Examples 4 and 9 to 15 containing phenolic compounds were excellent in gas barrier performance against the EOG sterilization treatment. The cured products obtained from the adhesives of Examples 4, 9, 11, and 12, which contain phenolic compounds having, as a substituent, an aliphatic hydrocarbon group (which may include an oxygen atom in a carbon-carbon bond) have demonstrated that the high gas barrier performance is maintained even after the EOG sterilization treatment is performed for 210 hours, and sufficient gas barrier performance is maintained even after hydrogen peroxide gas plasma sterilization is performed 100 cycles.

The present invention has been described together with embodiments thereof; however, we do not intend to limit our invention in any minor portion of the descriptions unless otherwise specified; we believe that the invention is construed broadly without departing from the spirit and scope of the invention described in the attached claims.

### REFERENCE SIGNS LIST

2 electronic endoscope (endoscope)

3 insertion section
3a flexible tube
3b angle portion
3c distal-end portion

5 main-body operation section

6 universal cord

11 spiral tube
11a metal strip

12 sleeve mesh body

13 metal cap

14 flexible-tube base
14a distal-end side
14b base-end side

15 resin layer

16 coating layer

17 adhesive-cured-product layer

31 illumination window

32 observation window

33 forceps port

34 nozzle

35 distal-end-portion main body

36 distal-end cap

37 lens holder

38 prism

39 substrate

40 solid image pickup element

41 adhesive cured product

42 adhesive cured product

43 observation unit

What is claimed is:

1. An adhesive for an endoscope, the adhesive comprising an epoxy resin including at least one of bisphenol A epoxy resin, bisphenol F epoxy resin, or phenol novolac epoxy resin, a curing component, and an acidic curing acceleration component.

2. The adhesive for an endoscope according to claim 1, wherein the acidic curing acceleration component is a compound having a phenolic hydroxy group.

3. The adhesive for an endoscope according to claim 1 wherein the curing component is a polyamine compound.

4. The adhesive for an endoscope according to claim 3, wherein the curing component is a polyether-polyamine compound.

5. The adhesive for an endoscope according to claim 1, being used as a sealing material.

6. A cured product provided by curing the adhesive for an endoscope according to claim 1.

7. An endoscope comprising a constituent member fixed using the cured product according to claim 6.

8. A method for producing an endoscope, the method comprising fixing a constituent member using the adhesive for an endoscope according to claim 1.
